(19) United States
(12) Patent Application Publication
(10) Pub. No.: US 2018/0014872 A1
(43) Pub. Date: Jan. 18, 2018

(54) PAIRED DEVICE AND GENERATOR CODES

(71) Applicant: Ethicon Endo-Surgery, LLC, Guaynabo, PR (US)

(72) Inventor: Benjamin D. Dickerson, Cincinnati, OH (US)

(21) Appl. No.: 15/212,141

(22) Filed: Jul. 15, 2016

(51) Int. Cl. A61B 18/12; A61B 18/14; A61B 17/32; A61B 17/00; A61B 18/00

(52) U.S. Cl. CPC A61B 18/1206; A61B 18/1445; A61B 17/320092; A61B 2018/00886; A61B 2018/00702; A61B 2018/00607; A61B 2017/00482; A61B 2018/00898; A61B 2018/00988; A61B 2018/00684

(57) ABSTRACT

A medical device energy source may comprise an energy supply, a power interface, a computing device, a networking interface, and a data interface. The power interface and the data interface may communicate with a medical device and the networking interface may communicate with a data server. The energy source may receive an identifier code from the medical device and receive medical device identity codes from the data server. The energy source may receive additional medical device information from the data server. The energy source may compare the identifier code with each of the identity codes and control the function of the energy supply based on the comparison of the identifier code with the identity codes. The energy source may also maintain usage records of the medical device. A medical device system may include the medical device energy source, the medical device, and the data server.

### BACKGROUND

Electrosurgical devices are used in many surgical procedures which may include removing, shrinking, or sealing tissues as part of the therapeutic process. In some examples, electrosurgical devices may apply electrical energy directly to tissue in order to effect the surgical treatment. Alternatively, electrosurgical devices may use the electrical energy as a source of power for other modes of surgical treatment, for example to generate ultrasonic energy which may then be applied to the tissues. An electrosurgical device may comprise an instrument having a distally-mounted end effector comprising components designed to introduce the therapeutic energy into the tissue being treated. Such end effectors may consist of two or more jaws in which at least one of the jaws is moveable from a position spaced apart from the opposing jaw for receiving tissues to a position in which the space between the jaws is less than that of the first position. Movement of the moveable jaw may compress the tissue held between. The therapeutic energy delivered by components of the end effector, in combination with the compression achieved by the jaw movement, may form hemostatic seals within the tissue and/or between tissues and thus may be particularly useful for sealing blood vessels, for example. The end effector of an electrosurgical device may also comprise a cutting member that is movable relative to the tissue and the jaws to transect the tissue.

In some electrosurgical devices, electrical energy may be transmitted to the instrument by a generator and applied directly by the electrosurgical device to the tissue under treatment. In some examples, the electrical energy may be in the form of radio frequency (“RF”) energy. The electrical energy may be in the form of radio frequency (“RF”) energy that may be in a frequency range described in EN 60601-2-2:2009+A11:20. In some applications, the applied energy may have a frequency restricted to less than 5 MHz. Typically, frequencies above 5 MHz are not used in order to minimize the problems associated with high frequency leakage currents. It is generally recognized that 10 mA is a lower threshold of thermal effects on tissue. RF energy may be supplied by a power source and introduced into tissue compressed between the two or more jaws. Such RF energy may cause ionic agitation in the tissue, in effect producing resistive heating, and thereby increasing the temperature of the tissue. Increased temperature of the tissue may lead to tissue cauterization. In some surgical procedures, RF energy may be useful for removing, shrinking, or sculpting soft tissue while simultaneously sealing blood vessels.

Other electrosurgical devices may use the electrical energy as a source of power for other modes of surgical treatment, for example to generate ultrasonic energy which may then be applied to the tissues. Ultrasonic surgical instruments can be used for the safe and effective treatment of many medical conditions. Generally, ultrasonic surgical instruments can be used to cut and/or coagulate organic tissue, for example, using energy in the form of ultrasonic vibrations, i.e., mechanical vibrations transmitted to a surgical end-effector at ultrasonic frequencies. These ultrasonic vibrations, when transmitted to organic tissue at suitable energy levels and using a suitable end-effector, may be used to cut and/or coagulate the tissue. Such instruments may be used for open procedures or minimally invasive procedures, such as endoscopic or laparoscopic procedures, for example, in which the end-effector of the medical device is passed through a trocar to reach a surgical site.

It may be recognized that proper control of the electrical energy supplied to an electrosurgical device is critical for safe and effective operation of the device. It is therefore desirable for a medical device energy source to supply an appropriate amount of electrical energy to an electrosurgical device to promote a safe and effective therapeutic outcome. For example, an appropriate amount of electrical energy may be dependent on the type of electrosurgical device and its usage history.

### SUMMARY

In one aspect, a medical device energy source may be composed of an energy source, an energy source power interface configured to deliver electrical energy from the energy source, and an energy source computing device. The energy source computing device may further be composed of an energy source processor unit, an energy source memory storage component in operative communication with the energy source processor unit, an energy source network communication interface in operative communication with the energy source processor unit, and an energy source data interface in operative communication with the energy source processor unit. The energy source computing device may be configured to control a function of the energy source. Further, the energy source memory storage component may include instructions that, when executed by the energy source processor unit, may cause the energy source computing device to receive an identifier code via the energy source data interface, receive a plurality of medical device identity codes via the energy source network communication interface, compare the identifier code with each of the plurality of medical device identity codes, and control the function of the energy source based on the comparison of the identifier code with each of the plurality of medical device identity codes.

In one aspect of the medical device energy source, the identifier code may be composed of two identifier strings, each of which may include a string of processor readable characters.

In one aspect of the medical device energy source, the instructions that cause the energy source computing device to compare the identifier code with each of the plurality of medical device identity codes may include instructions that cause the energy source computing device to compare each of two identifier strings with each of two identity strings comprising each of the medical device identity codes.

In one aspect of the medical device energy source, the energy source memory storage component may include instructions that, when executed by the energy source processor unit, further cause the energy source computing device to receive, via the energy source network communication interface, a plurality of medical device status indicators, in which each medical device status indicator corresponds to each of the plurality of medical device identity codes.

In one aspect of the medical device energy source, the instructions that cause the energy source computing device to control the function of the energy source further comprise instructions that cause the energy source computing device to control the function of the energy source base on the medical device status indicators corresponding to a medical device identity code equal to the identifier code.

In one aspect of the medical device energy source, the energy source memory storage component may include instructions that, when executed by the energy source processor unit, further cause the energy source computing device to retain, in the energy source memory storage component, an energizer value corresponding to an amount of energy supplied by the energy source, an energizer time value corresponding to a length of time during which the energy source supplies an amount of energy, an energizer number corresponding to a number of times the energy source supplies an amount of energy, or combinations thereof.

In one aspect, the medical device energy source may further include a user display in operative communication with the energy source processor unit.

In one aspect, a medical device system may be composed of a medical device, a medical device energy source, and a medical device network server. The medical device may be composed of a device memory storage component configured to store an identifier code; a device data interface in operative connection with the memory storage component; and a device power interface configured to receive electric power from an energy source. The medical device energy source may be composed of the energy source, an energy source power interface in operative communication with the device power interface and configured to deliver electrical energy from the energy source to the medical device, and an energy source computing device. The energy source computing device may further be composed of an energy source processor unit, an energy source memory storage component in operative communication with the energy source processor unit, an energy source network communication interface in operative communication with the energy source processor unit and configured to transmit data to and receive data from a communication network, and an energy source data interface in operative connection with the device data interface, in which the energy source computing device may be configured to control a function of the energy source. The medical device network server may be composed of a network server processor unit, a network server memory storage component in operative communication with the network server processor unit and configured to store a medical device database comprising a plurality of medical device identity codes and corresponding medical device status indicators, and a network server communication interface in operative communication with the network server processor unit and configured to transmit data to and receive data from at least one medical device power source via the communication network. In this aspect, the energy source memory storage component may include instructions that, when executed by the energy source processor unit, cause the energy source computing device to receive, from the device memory storage component, the identifier code, receive, from the network server memory storage component, the plurality of medical device identity codes from the medical device database, compare the identifier code with each of the plurality of medical device identity codes, and control the function of the energy source based on the comparison of the at least one identifier code with the plurality of medical device identity codes.

In one aspect of the medical device system, the identifier code may be composed of two identifier strings, each of which may include a string of processor readable characters. In this aspect, the instructions that cause the energy source computing device to compare the identifier code with each of the plurality of medical device identity codes includes instructions that cause the energy source computing device to compare each of the two identifier strings with each of two identity strings comprising each of the medical device identity codes.

In one aspect of the medical device system, the energy source memory storage component may include instructions that, when executed by the energy source processor unit, further cause the energy source computing device to receive, via the energy source network communication interface, a plurality of medical device status indicators, in which each of the plurality of medical device status indicators corresponds to each of the plurality of medical device identity codes. Further, the instructions that cause the energy source computing device to control the function of the energy source further include instructions that cause the energy source computing device to control the function of the energy source base on a medical device status indicator corresponding to a medical device identity code equal to the identifier code.

In one aspect of the medical device system, the energy source memory storage component may further include instructions that, when executed by the energy source processor unit, cause the energy source computing device to transmit, to the medical device network server, data to update a medical device status indicator corresponding to a medical device identity code equal to the identifier code.

In one aspect of the medical device system, the network server memory storage component may include instructions that, when executed by the network servicer processor unit, cause the network server processor unit to receive, from the medical device energy source, data to update a medical device status indicator corresponding to the medical device identity code equal to the identifier code and to update the status indicator in the data base corresponding to the medical device identity code equal to the identifier code.

In one aspect of the medical device system, the medical device data base may further include one or more additional indicators corresponding to each of the medical device identity codes in the medical device data base.

In one aspect of the medical device system, the energy source memory storage component may further include instructions that, when executed by the energy source processor unit, cause the energy source computing device to store in the energy source memory storage component an indicator of total medical device uses and a total amount of power supplied by the medical device energy source to the medical device over the total number of medical device uses. The energy source memory storage component may further include instructions that, when executed by the energy source processor unit, cause the energy source computing device to store in the energy source memory storage component an indicator, for each use of the total medical device uses, of an amount of power supplied by the medical device energy source to the medical device, and a length of time during which the medical device energy source supplies the amount of energy to the medical device.

In one aspect of the medical device system, the energy source memory storage component may further include instructions that, when executed by the energy source processor unit, cause the energy source computing device to receive, from the medical device network server, values of the one or more additional indicators corresponding to each of the medical device identity codes in the medical device data base and control the function of the energy source based on the value of the one or more of the additional indicators corresponding to the medical device identity code equal to the identifier code.

In one aspect of the medical device system, he network server memory storage component may include instructions that, when executed by the network servicer processor unit, cause the network server processor unit to receive, from the medical device energy source, values of the one or more additional indicators corresponding to each of the medical device identity codes in the medical device data base, and update the values of the one or more additional indicators corresponding to each of the medical device identity codes in the medical device database.

In one aspect, a method of controlling a medical device may include receiving, by a medical device energy source via an energy source data interface in operative communication with an energy source processor unit, an identifier code from a medical device, storing, by the medical device energy source in an energy source memory storage component in operative communication with the energy source processor unit, the identifier code, receiving, by the medical device energy source via an energy source network communication interface in operative communication with the energy source processor unit, a plurality of medical device identity codes from a medical device network server, comparing, by the energy source processor unit, the identifier code with each of the plurality of medical device identity codes, controlling, by the energy source processor unit, an amount of energy delivered by the energy source via an energy source power interface to the medical device, based on the comparison between the identifier code and the plurality of medical device identity codes, and displaying, on a user display operatively controlled by an energy source computing device comprising the energy source processor unit, information corresponding to the amount of energy delivered by the energy source to the medical device.

In one aspect, the method may further include transmitting, by the medical device energy source to the medical device, control data to control at least one function of the medical device.

In one aspect, the method may further include receiving, by the medical device energy source via the energy source network communication interface, a plurality of medical device status indicators, wherein each of the plurality of medical device status indicators corresponds to each of the plurality of medical device identity codes.

In one aspect, the method may further include controlling, by the energy source processor unit, an amount of energy delivered by the energy source via the energy source power interface to the medical device, based on the medical device status indicator corresponding to a medical device identity code that is equal to the identifier code.

In one aspect, the method may further include transmitting, by the medical device energy source to the medical device, control data to control at least one function of the medical device based on the medical device status indicators corresponding to the medical device identity code that is equal to the identifier code.

### BRIEF DESCRIPTION OF THE FIGURES

The features of the various aspects are set forth with particularity in the appended claims. The various aspects, however, both as to organization and methods of operation, together with advantages thereof, may best be understood by reference to the following description, taken in conjunction with the accompanying drawings as follows:

FIG. 1A illustrates one form of a surgical system comprising a generator and various surgical instruments usable therewith.

FIG. 1B is a diagram of the ultrasonic surgical instrument of FIG. 1.

FIG. 1C is a diagram of the surgical system of FIG. 1.

FIG. 2 illustrates a block diagram of an example of a medical device system.

FIGS. 3A and 3B illustrate block diagrams of examples of medical devices which may be a component of the medical device system illustrated in FIG. 2.

FIGS. 4A and 4B illustrate block diagrams of examples of medical device energy sources which may be a component of the medical device system illustrated in FIG. 2.

FIG. 5 illustrates a block diagram of an example of a medical device network server which may be a component of the medical device system illustrated in FIG. 2.

FIGS. 6A-6D illustrate examples of data structures in a database which may be stored in a memory component of the medical device network server illustrated in FIG. 5.

FIGS. 7A and 7B are simplified flow charts illustrating programming a medical device for use in the medical device system illustrated in FIG. 2.

FIGS. 8A and 8B are simplified flow charts illustrating using a medical device which is part of the medical device system illustrated in FIG. 2.

### DETAILED DESCRIPTION

Reference will now be made in detail to several aspects, including example implementations of electrosurgical medical instruments for cutting and coagulating tissue. Wherever practicable similar or like reference numbers may be used in the figures and may indicate similar or like functionality. The figures depict examples of the disclosed surgical instruments and/or methods of use for purposes of illustration only. One skilled in the art will readily recognize from the following description that alternative examples of the structures and methods illustrated herein may be employed without departing from the principles described herein.

Various aspects of surgical instruments that utilize therapeutic and/or sub-therapeutic electrical energy to treat tissue or provide feedback to the generators (e.g., electrosurgical instruments). The various aspects are adapted for use in a manual or hand operated manner, although electrosurgical instruments may be utilized in robotic applications as well.

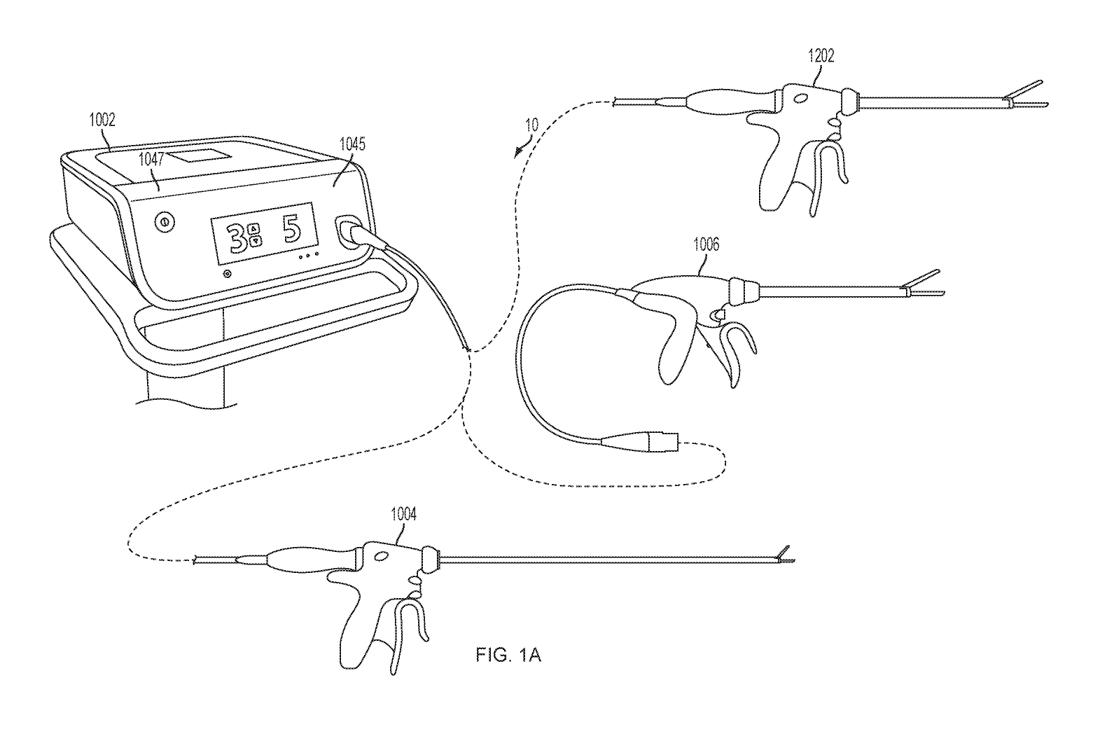

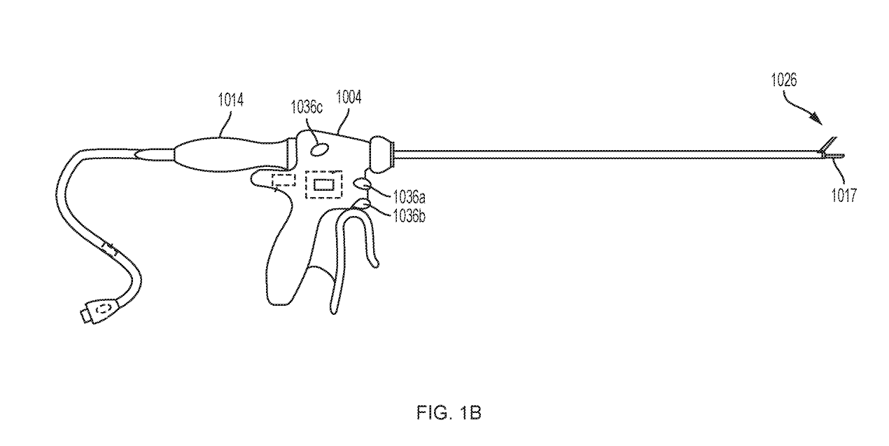

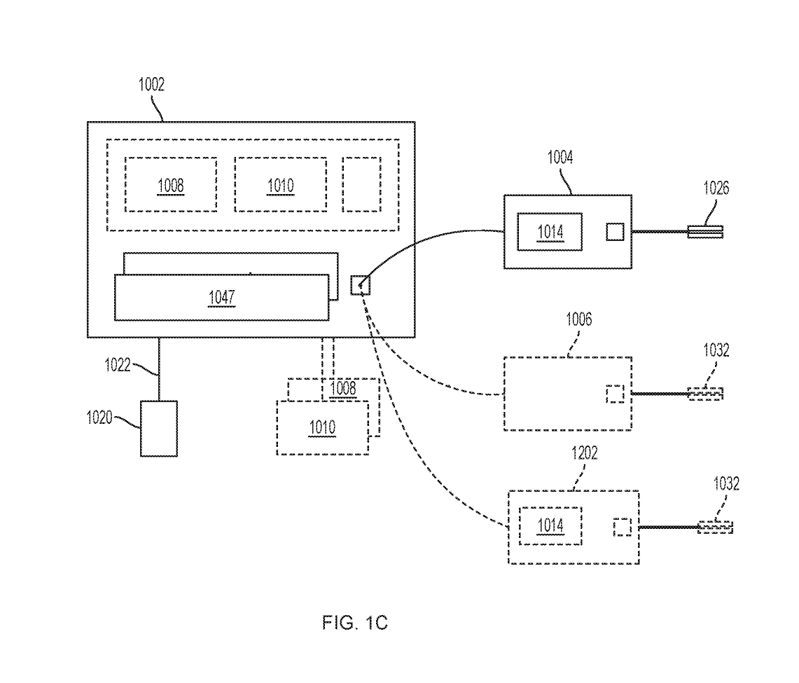

With reference to FIGS. 1A-1C, one form of a surgical system 10 including an ultrasonic surgical instrument is illustrated. FIG. 1A illustrates one form of a surgical system 10 comprising a generator 1002 and various surgical instruments 1004, 1006, 1202 usable therewith. FIG. 1B is a diagram of the ultrasonic surgical instrument 1004 of FIG. 1A. With reference to both FIGS. 1A and 1B, the generator 1002 is configurable for use with a variety of surgical devices. According to various forms, the generator 1002 may be configurable for use with different surgical devices of different types including, for example, the ultrasonic device 1004, electrosurgical or RF surgical devices, such as, the RF device 1006, and multifunction devices 1202 that integrate electrosurgical RF and ultrasonic energies delivered simultaneously from the generator 1002. Although in the form of FIG. 1A, the generator 1002 is shown separate from the surgical devices 1004, 1006, 1202 in one form, the generator 1002 may be formed integrally with either of the surgical devices 1004, 1006, 1202 to form a unitary surgical system. The generator 1002 comprises an input device 1045 located on a front panel of the generator 1002 console. The input device 1045 may comprise any suitable device that generates signals suitable for programming the operation of the generator 1002.

FIG. 1C is a diagram of the surgical system 10 of FIG. 1A. In various forms, the generator 1002 may comprise several separate functional elements, such as modules and/or blocks. Different functional elements or modules may be configured for driving the different kinds of surgical devices 1004, 1006, 1202. For example, an ultrasonic generator module 1008 may drive ultrasonic devices such as the ultrasonic device 1004. An electrosurgery/RF generator module 1010 may drive the electrosurgical device 1006. For example, the respective modules 1008, 1010 may generate respective drive signals for driving the surgical devices 1004, 1006, 1202. In various forms, the ultrasonic generator module 1008 and/or the electrosurgery/RF generator module 1010 each may be formed integrally with the generator 1002. Alternatively, one or more of the modules 1008, 1010 may be provided as a separate circuit module electrically coupled to the generator 1002. (The modules 1008 and 1010 are shown in phantom to illustrate this option.) Also, in some forms, the electrosurgery/RF generator module 1010 may be formed integrally with the ultrasonic generator module 1008, or vice versa. Also, in some forms, the generator 1002 may be omitted entirely and the modules 1008, 1010 may be executed by processors or other hardware within the respective instruments 1004, 1006, 1202.

In other forms, the electrical outputs of the ultrasonic generator module 1008 and the electrosurgery/RF generator module 1010 may be combined into a single electrical signal capable of driving the multifunction device 1202 simultaneously with electrosurgical RF and ultrasonic energies. The multifunction device 1202 comprises an ultrasonic transducer 1014 coupled to an ultrasonic blade and one or more electrodes in the end effector 1032 to receive electrosurgical RF energy. In such implementations, the combined RF/ultrasonic signal is coupled to the multifunction device 1202. The multifunction device 1202 comprises signal processing components to split the combined RF/ultrasonic signal such that the RF signal can be delivered to the electrodes in the end effector 1032 and the ultrasonic signal can be delivered to the ultrasonic transducer 1014.

In accordance with the described forms, the ultrasonic generator module 1008 may produce a drive signal or signals of particular voltages, currents, and frequencies, e.g., 55,500 cycles per second (Hz). The drive signal or signals may be provided to the ultrasonic device 1004, and specifically to the transducer 1014, which may operate, for example, as described above. The transducer 1014 and a waveguide extending through the shaft (waveguide not shown in FIG. 1B) may collectively form an ultrasonic drive system driving an ultrasonic blade 1017 of an end effector 1026. In one form, the generator 1002 may be configured to produce a drive signal of a particular voltage, current, and/or frequency output signal that can be stepped or otherwise modified with high resolution, accuracy, and repeatability.

The generator 1002 may be activated to provide the drive signal to the transducer 1014 in any suitable manner. For example, the generator 1002 may comprise a foot switch 1020 coupled to the generator 1002 via a footswitch cable 1022. A clinician may activate the transducer 1014 by depressing the foot switch 1020. In addition, or instead of the foot switch 1020 some forms of the ultrasonic device 1004 may utilize one or more switches positioned on the hand piece that, when activated, may cause the generator 1002 to activate the transducer 1014. In one form, for example, the one or more switches may comprise a pair of toggle buttons 1036a, 1036b (FIG. 1B), for example, to determine an operating mode of the device 1004. When the toggle button 1036a is depressed, for example, the ultrasonic generator 1002 may provide a maximum drive signal to the transducer 1014, causing it to produce maximum ultrasonic energy output. Depressing toggle button 1036b may cause the ultrasonic generator 1002 to provide a user-selectable drive signal to the transducer 1014, causing it to produce less than the maximum ultrasonic energy output. The device 1004 additionally or alternatively may comprise a second switch (not shown) to, for example, indicate a position of a jaw closure trigger for operating jaws of the end effector 1026. Also, in some forms, the ultrasonic generator 1002 may be activated based on the position of the jaw closure trigger, (e.g., as the clinician depresses the jaw closure trigger to close the jaws, ultrasonic energy may be applied).

Additionally or alternatively, the one or more switches may comprise a toggle button 1036c that, when depressed, causes the generator 1002 to provide a pulsed output. The pulses may be provided at any suitable frequency and grouping, for example. In certain forms, the power level of the pulses may be the power levels associated with toggle buttons 1036a, 1036b (maximum, less than maximum), for example.

It will be appreciated that a device 1004 may comprise any combination of the toggle buttons 1036a, 1036b, 1036c. For example, the device 1004 could be configured to have only two toggle buttons: a toggle button 1036a for producing maximum ultrasonic energy output and a toggle button 1036c for producing a pulsed output at either the maximum or less than maximum power level. In this way, the drive signal output configuration of the generator 1002 could be 5 continuous signals and 5 or 4 or 3 or 2 or 1 pulsed signals. In certain forms, the specific drive signal configuration may be controlled based upon, for example, EEPROM settings in the generator 1002 and/or user power level selection(s).

In certain forms, a two-position switch may be provided as an alternative to a toggle button 1036c. For example, a device 1004 may include a toggle button 1036a for producing a continuous output at a maximum power level and a two-position toggle button 1036b. In a first detented position, toggle button 1036b may produce a continuous output at a less than maximum power level, and in a second detented position the toggle button 1036b may produce a pulsed output (e.g., at either a maximum or less than maximum power level, depending upon the EEPROM settings).

In accordance with the described forms, the electrosurgery/RF generator module 1010 may generate a drive signal or signals with output power sufficient to perform bipolar electrosurgery using radio frequency (RF) energy. In bipolar electrosurgery applications, the drive signal may be provided, for example, to electrodes of the electrosurgical device 1006, for example. Accordingly, the generator 1002 may be configured for therapeutic purposes by applying electrical energy to the tissue sufficient for treating the tissue (e.g., coagulation, cauterization, tissue welding).

The generator 1002 may comprise an input device 1045 (FIG. 1A) located, for example, on a front panel of the generator 1002 console. The input device 1045 may comprise any suitable device that generates signals suitable for programming the operation of the generator 1002. In operation, the user can program or otherwise control operation of the generator 1002 using the input device 1045. The input device 1045 may comprise any suitable device that generates signals that can be used by the generator (e.g., by one or more processors contained in the generator) to control the operation of the generator 1002 (e.g., operation of the ultrasonic generator module 1008 and/or electrosurgery/RF generator module 1010). In various forms, the input device 1045 includes one or more of buttons, switches, thumbwheels, keyboard, keypad, touch screen monitor, pointing device, remote connection to a general purpose or dedicated computer. In other forms, the input device 1045 may comprise a suitable user interface, such as one or more user interface screens displayed on a touch screen monitor, for example. Accordingly, by way of the input device 1045, the user can set or program various operating parameters of the generator, such as, for example, current (I), voltage (V), frequency (f), and/or period (T) of a drive signal or signals generated by the ultrasonic generator module 1008 and/or electrosurgery/RF generator module 1010.

The generator 1002 may also comprise an output device 1047 (FIG. 1A), such as an output indicator, located, for example, on a front panel of the generator 1002 console. The output device 1047 includes one or more devices for providing a sensory feedback to a user. Such devices may comprise, for example, visual feedback devices (e.g., a visual feedback device may comprise incandescent lamps, light emitting diodes (LEDs), graphical user interface, display, analog indicator, digital indicator, bar graph display, digital alphanumeric display, LCD display screen, LED indicators), audio feedback devices (e.g., an audio feedback device may comprise speaker, buzzer, audible, computer generated tone, computerized speech, voice user interface (VUI) to interact with computers through a voice/speech platform), or tactile feedback devices (e.g., a tactile feedback device comprises any type of vibratory feedback, haptic actuator).

Although certain modules and/or blocks of the generator 1002 may be described by way of example, it can be appreciated that a greater or lesser number of modules and/or blocks may be used and still fall within the scope of the forms. Further, although various forms may be described in terms of modules and/or blocks to facilitate description, such modules and/or blocks may be implemented by one or more hardware components, e.g., processors, Digital Signal Processors (DSPs), Programmable Logic Devices (PLDs), Application Specific Integrated Circuits (ASICs), circuits, registers and/or software components, e.g., programs, subroutines, logic and/or combinations of hardware and software components. Also, in some forms, the various modules described herein may be implemented utilizing similar hardware positioned within the instruments 1004, 1006, 1202 (i.e., the generator 1002 may be omitted).

In one form, the ultrasonic generator drive module 1008 and electrosurgery/RF drive module 1010 may comprise one or more embedded applications implemented as firmware, software, hardware, or any combination thereof. The modules 1008, 1010 may comprise various executable modules such as software, programs, data, drivers, application program interfaces (APIs), and so forth. The firmware may be stored in nonvolatile memory (NVM), such as in bit-masked read-only memory (ROM) or flash memory. In various implementations, storing the firmware in ROM may preserve flash memory. The NVM may comprise other types of memory including, for example, programmable ROM (PROM), erasable programmable ROM (EPROM), electrically erasable programmable ROM (EEPROM), or battery backed random-access memory (RAM) such as dynamic RAM (DRAM), Double-Data-Rate DRAM (DDRAM), and/or synchronous DRAM (SDRAM).

In one form, the modules 1008, 1010 comprise a hardware component implemented as a processor for executing program instructions for monitoring various measurable characteristics of the devices 1004, 1006, 1202 and generating a corresponding output control signals for operating the devices 1004, 1006, 1202. In forms in which the generator 1002 is used in conjunction with the device 1004, the output control signal may drive the ultrasonic transducer 1014 in cutting and/or coagulation operating modes. Electrical characteristics of the device 1004 and/or tissue may be measured and used to control operational aspects of the generator 1002 and/or provided as feedback to the user. In forms in which the generator 1002 is used in conjunction with the device 1006, the output control signal may supply electrical energy (e.g., RF energy) to the end effector 1032 in cutting, coagulation and/or desiccation modes. Electrical characteristics of the device 1006 and/or tissue may be measured and used to control operational aspects of the generator 1002 and/or provide feedback to the user. In various forms, as previously discussed, the hardware component may be implemented as a DSP, PLD, ASIC, circuits, and/or registers. In one form, the processor may be configured to store and execute computer software program instructions to generate the step function output signals for driving various components of the devices 1004, 1006, 1202, such as the ultrasonic transducer 1014 and the end effectors 1026, 1032.

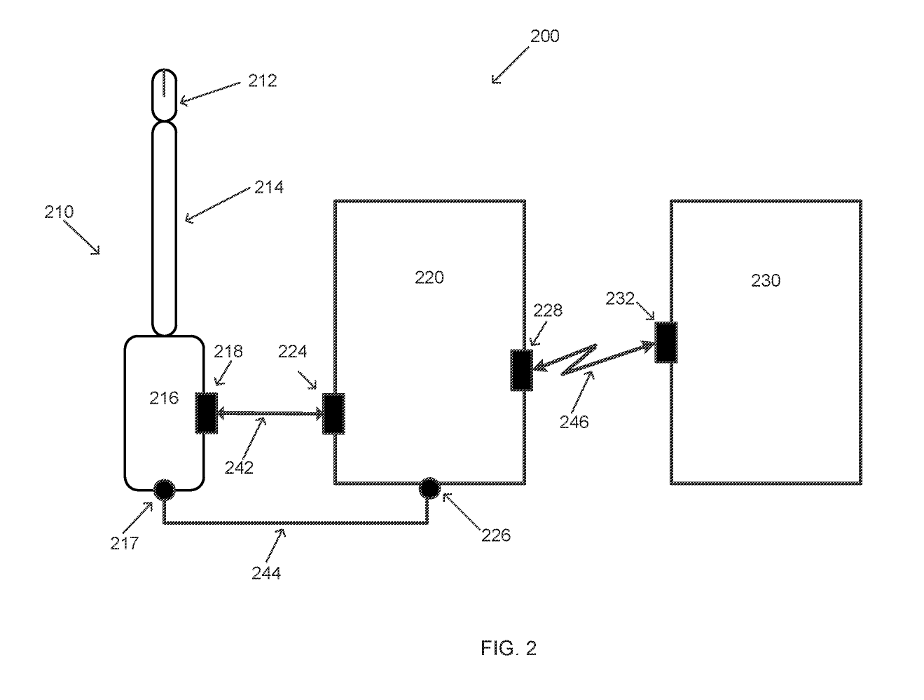

FIG. 2 illustrates a medical device system 200 that may include an electrosurgical medical device 210 and a medical device energy source 220. The medical device 210 may include a hand-held component 216, an end effector 212, and an introducer or elongated shaft 214. The medical device 210 may also include a device data interface 218 and a device power interface 217. The medical device energy source 220 may also include complementary interfaces, including an energy source power interface 226 and an energy source data interface 224.

The energy source power interface 226 may be configured to source electrical energy to the device power interface 217. In some non-limiting examples, the electrical energy may be transmitted from the medical device energy source 220 to the medical device 210 by means of a power cable 244. Further, the energy source data interface 224 may be configured to receive data from or transmit data to the device data interface 218. Such data may be used by the medical device 210 to control one or more medical device functions. Alternatively, data from the medical device 210 may be transmitted from the device data interface 218 to the energy source data interface 224. The data from the medical device 210 may be stored by the energy source 220 or may be used by the energy source 220 to control one or more energy source functions. In some non-limiting examples, the data transmitted from the medical device energy source 220 to the medical device 210, or by the medical device 210 to the energy source 220, may be accomplished by means of a data cable 242.

In some non-limiting examples, the medical device data interface 218 and the energy source data interface 224 may include wireless interfaces. Such wireless interfaces may not require a data cable 242 for exchanging data between the energy source 220 and the medical device 210.

In some non-limiting examples, the medical device data interface 218 and the medical device power interface 217 may be merged into a single medical device interface. Similarly, the energy source power interface 226 and the energy source data interface 224 may be merged into a single energy source interface. In such examples, a single power/data cable may be capable of transmitting both electrical energy and data.

The medical device system 200 may also include a medical device network server 230 having a network server communication interface 232. The medical device network server 230 may store one or more databases of information related to the medical device 210, the energy source 220, and their respective functions. The medical device network server 230 may be in data communication with the energy source 220 via a network server communication interface 232 and an energy source network communication interface 228. Data communication 246 between the medical device network server 230 and the energy source 220 may be accomplished through any standard data exchange method. Thus, the data communication 246 may include parallel communications methods, serial communications methods, optical communications methods, internet communications methods, wireless communication methods, and cellular communication methods. Although the medical device network server 230 is depicted in FIG. 2 as a single device, it may be recognized that the medical device network server 230 may comprise distributed servers, a cloud-based server, or other server configurations that may store the database.

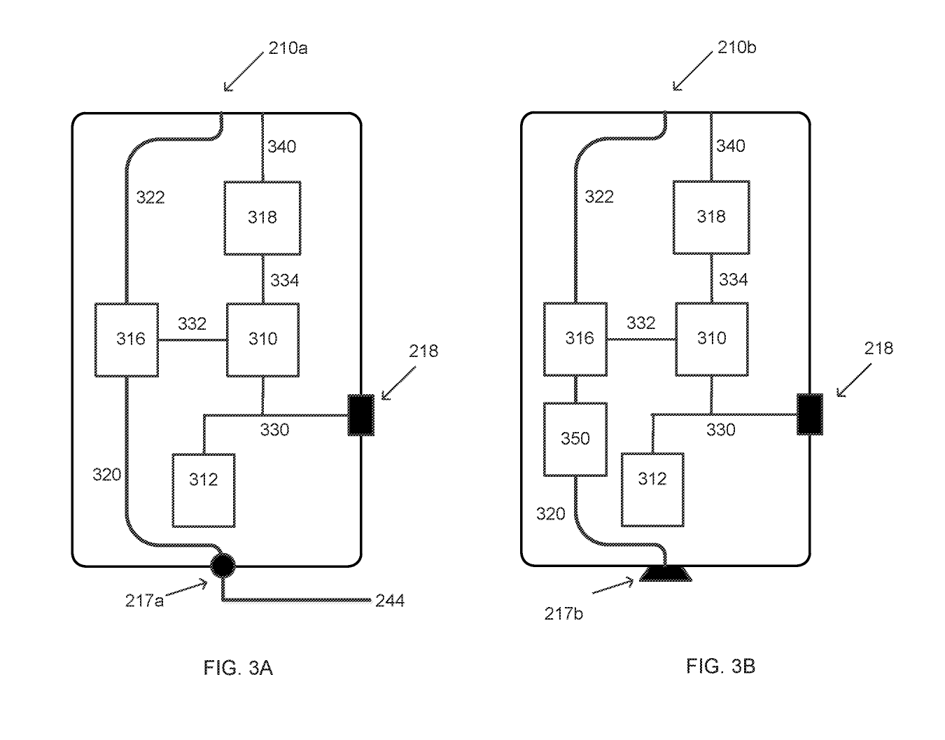

FIGS. 3A-B, 4A-B, and 5, disclosed below, depict exemplary components of the medical device system 200 greater detail. It may be understood that a medical device system 200 may not be limited to the specific components depicted in FIGS. 3A-B, 4A-B, and 5 and as disclosed below but may include additional components or lack certain components as disclosed.

FIGS. 3A and 3B depict two examples of a portion of a medical device, 210a and 210b, respectively. It may be recognized that the parts depicted in FIGS. 3A and 3B may be incorporated in the medical device hand-held component 216 or may be distributed throughout the medical device 210 as required.

FIG. 3A depicts a portion of a medical device 210a having a device power interface 217a configured to accept a power cable 244. A device energy controller 316 may receive electrical energy from the device power interface 217a through a device power bus 320. The device energy controller 316 may be configured to regulate or control electrical power delivered to additional components of the medical device 210a via a secondary device power bus 322. In one non-limiting example, the device energy controller 316 may direct electrical energy to one or more electrodes at the medical device end effector (1032 in FIG. 10) for direct energy—for example, RF energy—application to a tissue. In another non-limiting example, the device energy controller 316 may direct electrical energy to one or more additional components, such as a piezoelectric component, configured to convert electrical energy to ultrasound energy. It may be recognized that the electrical energy received by the medical device 210a, 210b may also be used to power a variety of electrical and/or electromechanical components found therein.

In general, the medical device 210a, 201b may comprise various physical or logical elements implemented as hardware, software, or any combination thereof, as desired for a given set of design parameters or performance constraints. In various aspects, the physical or logical elements may be connected by one or more communications media. For example, communication media may comprise wired communication media (including one or more communication busses), wireless communication media, or a combination of both, as desired for a given implementation.

The medical device 210a, 201b further comprises a device processor unit 310 and one or more device memory storage components 312. The device processor unit 310 and the one or more memory storage components 312 may be in data communication via a device data bus 330. The device data interface 218 may also be in data communication with the processor unit 310 and the one or more memory storage components 312 via the device data bus 330. The device processor unit 310 may also be in communication with the device energy controller 316 over an energy control bus 332. Alternatively, the device energy controller 316 may be in communication with the device processor unit 310 via the device data bus 330.

The device data interface 218 may include any data communication interface that may be in data communication with the medical device energy source 220. Such an interface may be a wired interface or a wireless interface. Wired communication modes include any mode of communication between points that utilizes wired technology including various protocols and combinations of protocols associated with wired transmission, data, and devices. Wireless communication modes include any mode of communication between points that utilizes, at least in part, wireless technology including various protocols and combinations of protocols associated with wireless transmission, data, and devices. Non-limiting examples of wired communication interfaces may include a serial interface, a parallel interface, an ethernet interface, and an optical cable interface. Non-limiting examples of a wireless interface may include a wireless local area network (WLAN) interface, a wireless wide area network (WWAN) interface, and a wireless personal area network (PAN) interface.

The device processor unit 310 may also control one or more electromechanical components 318 via one or more additional control lines 334. The one or more electromechanical components 318 may include relays, motors, or other components configured to convert electrical energy into mechanical actuation. The mechanical actuation of the electromechanical components 318 may be transmitted via mechanical linkages 340 to other mechanical components of the medical device 210a, 210b such as jaw actuators and cutting actuators at the end effector (1032, 1026 in FIG. 10).

The device processor unit 310 may also be in communication with one or more input and/or output interfaces of the medical device 210a, 201b. Input interfaces may include, without limitation, push buttons, slide buttons, pressure sensors, heat sensors, magnetic sensors, light sensors, or other inputs associated with the medical device 210a, 201b which may provide data regarding device use. Output interfaces may be used to activate, without limitation, LEDs, LED displays, LCD displays, audio indicators, haptic indicators, or other indicators to notify the user of the status of the medical device 210a, 201b.

The device processor unit 310 may be implemented as a general purpose processor, a chip multiprocessor (CMP), a dedicated processor, an embedded processor, a digital signal processor (DSP), a microprocessor such as a complex instruction set computer (CISC) microprocessor, a reduced instruction set computing (RISC) microprocessor, and/or a very long instruction word (VLIW) microprocessor, or other processing device. The device processor unit 310 also may be implemented by a controller, a microcontroller, an application specific integrated circuit (ASIC), a field programmable gate array (FPGA), a programmable logic device (PLD), and so forth. In various aspects, the device processor unit 310 may be arranged to run an operating system (OS) and various mobile applications. Examples of an OS include, for example, operating systems generally known under the trade name of Microsoft Windows OS, and any other proprietary or open source OS.

In various aspects, the one or more device memory storage components 312 may comprise any machine-readable or computer-readable media capable of storing data, including both volatile and non-volatile memory. For example, memory may include read-only memory (ROM), random-access memory (RAM), dynamic RAM (DRAM), Double-Data-Rate DRAM (DDR-RAM), synchronous DRAM (SDRAM), static RAM (SRAM), programmable ROM (PROM), erasable programmable ROM (EPROM), electrically erasable programmable ROM (EEPROM), flash memory (e.g., NOR or NAND flash memory), content addressable memory (CAM), polymer memory (e.g., ferroelectric polymer memory), phase-change memory (e.g., ovonic memory), ferroelectric memory, silicon-oxide-nitride-oxide-silicon (SONOS) memory, or any other type of media suitable for storing information.

The device data bus 330 and the energy control bus 332 may be composed of any of several types of bus structure(s) including the memory bus or memory controller, a peripheral bus or external bus, and/or a local bus using any variety of available bus architectures including, but not limited to, 9-bit bus, Industrial Standard Architecture (ISA), Micro-Charmel Architecture (MSA), Extended ISA (EISA), Intelligent Drive Electronics (IDE), VESA Local Bus (VLB), Peripheral Component Interconnect (PCI), Universal Serial Bus (USB), Advanced Graphics Port (AGP), Personal Computer Memory Card International Association bus (PCMCIA), Small Computer Systems Interface (SCSI) or other proprietary bus.

The one or more device memory storage components 312 may be used to store instructions that may be executed by the device processor unit 310. The instructions may include any suitable type of code, such as source code, compiled code, interpreted code, executable code, static code, dynamic code, and the like. The instruction or a set of instructions may include those that, if executed by the device processor unit 310, may cause the device processor unit 310 to perform a method and/or operations in accordance with the embodiments. The instructions may be implemented using any suitable high-level, low-level, object-oriented, visual, compiled and/or interpreted programming language, such as C, C++, Java, BASIC, Perl, Matlab, Pascal, Visual BASIC, assembly language, machine code, and so forth.

Instructions stored in the one or more device memory storage components 312 may include instructions to control an amount of energy delivered by the device energy controller 316 to the end effector 212. Other instructions may control operations of the one or more electromechanical components 318. Additional instructions may cause the device processor unit 310 to store information associated with the use of the medical device 210a, 201b. Use information may include, without limitation, a number of times energy is delivered by the end effector 212 to a single piece of tissue or to multiple pieces of tissue, data related to an amount of energy delivered to a tissue by the end effector 212 for each application of energy to the tissue, and a length of time during with the amount of energy is delivered to the tissue. Additional use information may be stored, including, without limitation, temperature of a tissue receiving energy and an impedance measurement of the tissue.

The one or more device memory storage components 312 may also store data associated with the medical device 210a, 201b and its use. As disclosed above, data associated with device use may include, without limitation, a number of times energy is delivered by the end effector 212 to a single piece of tissue or to multiple pieces of tissue, data related to an amount of energy delivered to a tissue by the end effector 212 for each application of energy to the tissue, and a length of time during with the amount of energy is delivered to the tissue. Additional use information may be stored, including, without limitation, temperature of a tissue receiving energy and an impedance measurement of the tissue.

The one or more device memory storage components 312 may also store information characterizing the medical device 210a, 201b. Such characterizing information may include, without limitation, a device name, a device model number, a device lot or serial number, a device use limitation number, a device power limitation number, a device expiration date, and a device identifier code. The device identifier code may include one or more machine readable characters, or one or more strings of such machine readable characters. The device identifier code may be composed of a single string of machine readable characters. Alternatively, the device identifier code may be compose of a plurality of strings of machine readable characters, such as, as non-limiting examples, two strings of machine readable characters or three strings of machine readable characters. In one non-limiting example, the device identifier code may comprise a string of machine readable characters related to the device characterizing information. In another non-limiting example, the device identifier code may comprise one or more strings of randomly generated machine readable characters. The device identifier code may be composed of one or more strings of any number of machine readable characters. Non-limiting examples of the number of machine readable characters in each of the one or more strings of the device identifier code may include 32 characters, 64 characters, 128 characters, 256 characters, 512 characters, or any number of characters therebetween including endpoints.

FIG. 3B depicts a portion of a medical device 210b having a device power interface 217b configured to interface with a medical device energy source through an energy docking port. The medical device 210b having a docking power interface 217b may be operated free of a power cable 244 for easier handling. It may be observed that many of the components of medical device 210b are identical to those of medical device 210a as disclosed above. A cordless medical device 210b may include a rechargeable battery 350 to receive and store power received from a medical device energy source 220 while the medical device 210b is physically docked to the medical device energy source 220. Power from the battery 350 may be regulated through the device energy controller 316 in a manner suitable for power use for such a cordless medical device 210b. Data communication between the cordless medical device 210b and a medical device energy source 220 may be accomplished through a device data interface 218 which may be composed of a wired communication interface or a wireless communication interface as disclosed above with respect to a corded medical device 210a.

It should be understood that a corded medical device 210a that accepts electrical energy via a power cable 244 may also include a battery for electrical power storage. Such additional power storage capability may be used as a separate power source for electrical and/or electromechanical components of the medical device 210a. Such a battery may be used to electrically isolate the electrical or electronic components from noise on the device power bus 320 during the operation of the device. Alternatively, such a battery may serve as a back-up power supply to the electronic components in the event of a power failure of the medical device energy source 220.

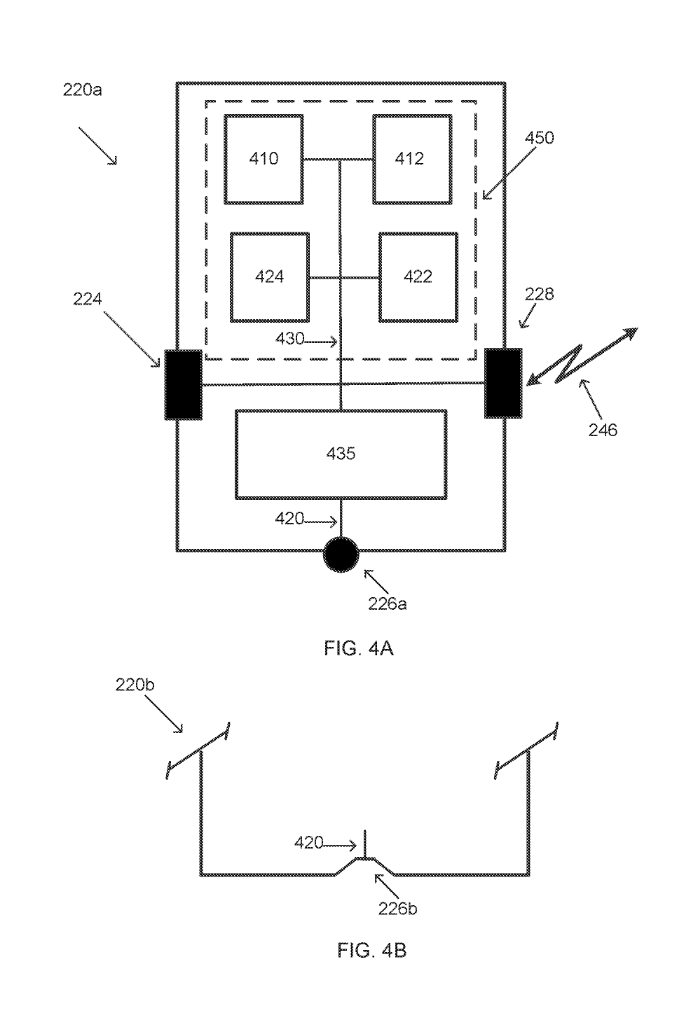

FIGS. 4A and 4B depict two examples of a medical device energy source, 220a and 220b, respectively. FIG. 4A depicts an example of a medical device energy source 220a that may be used with a corded medical device 210a. FIG. 4B depicts a portion of an exemplary medical device energy source 220b that may be used with a cordless medical device 210b.

As depicted in FIG. 4A, a medical device energy source 220a may incorporate an energy source 435 in electrical communication via an energy source device power bus 420 to an energy source power interface 226a configured to deliver electrical energy from the energy source. Energy source power interface 226a may be suitable for an attached power cable 244 configured to conduct electrical energy to a corded medical device 210a. The energy source 435 may be controlled to supply an effective amount of electrical energy to the medical device 210. An effective amount of electrical energy may comprise a therapeutic amount of energy, a non-therapeutic amount of energy, or both a therapeutic and a non-therapeutic amount of energy to the medical device. Non-limiting examples of a therapeutic amount of energy may include an amount of energy required to effect a therapy on a tissue, such as an amount of energy to cauterize a tissue, an amount of energy to shrink a tissue, or an amount of energy to cut a tissue according to the type of medical device 210 receiving the electrical energy. Non-limiting examples of a non-therapeutic amount of energy may include an amount of energy that is not sufficient to effect a therapy on a tissue including an amount of energy to measure a tissue impedance or an amount of energy to power electronic components of the medical device 210a. The electrical energy sourced by the energy source 435 may be controlled with respect to a DC voltage, an AC voltage, an RMS voltage, a DC current, an AC current, an RMS current, a frequency, a pulse-width modulation, or any combination thereof.

The energy source 435 may be controlled by an energy source computing device 450. The energy source computing device 450 may be composed of an energy source processor unit 410, one or more energy source memory storage components 412, one or more energy source input interfaces 424, one or more energy source output interfaces 422, and an energy source data bus 430 It may be understood that the energy source data bus 430 may be configured to place the one or more of the energy source memory storage components 412, one or more energy source input interfaces 424, and one or more energy source output interfaces 422 in operative communication with the energy source processor unit 410.

The one or more energy source memory storage components 412 may be used to store instructions that may be executed by the energy source processor unit 410. Some non-limiting examples of such instructions may include: instructions to receive data from the one or more energy source input interfaces 424; instructions related to a display of information on display devices that may be in operative communication with the energy source output interfaces 422; instructions to control the operation of the energy source 435; instructions to transmit data to the medical device via the energy source data interface 224; instructions to receive data from the medical device via the energy source data interface 224; instructions to determine that the medical device 210 is in functional communication with the medical device energy source 220; instructions that the energy source power interface 226a,b in operative communication with the device power interface 217a,b; and instructions to determine that the energy source data interface 224 is in operative communication with the device data interface 218. Additional instructions may cause the energy source 435 not to deliver an effective amount of electrical energy via the energy source power interface 226a,b to the medical device 210a,b when the medical device is not in functional communication with the medical device energy source. The instructions may include any suitable type of code, such as source code, compiled code, interpreted code, executable code, static code, dynamic code, and the like. The instruction or a set of instructions may include those that, if executed by the energy source processor unit 410, may cause the energy source processor unit 410 to perform a method and/or operations in accordance with the embodiments. The instructions may be implemented using any suitable high-level, low-level, object-oriented, visual, compiled and/or interpreted programming language, such as C, C++, Java, BASIC, Perl, Matlab, Pascal, Visual BASIC, assembly language, machine code, and so forth.

The energy source input interfaces 424 may comprise any interface configured to provide input from a user to the energy source computing device 450 via energy source data bus 430. Non-limiting examples of such an input interface 424 may include a serial interface, a parallel interface, an audio (microphone) interface, a wireless interface including an RF interface, and an optical interface. Such an energy source input interface 424 may be in data communication with any number of user actuators including, without limitation, push buttons, slide buttons, keyboards, knobs, touch screens, and computer mice. A user may employ the actuators to direct the operation of the medical device energy source 220a, for example setting a maximum amount of electrical energy to be supplied by the energy source 435, or a number of times the energy source 435 may supply electrical energy to the medical device 210a.

The energy source output interface 422 may comprise any interface configured to provide information to a user to the energy source computing device 450 via energy source data bus 430. The user display may thus be in operative communication with the energy source processor unit 410 by means of the energy source output interface 422 and the energy source bus 430. Non-limiting examples of such an output interface 422 may include a serial interface, a parallel interface, a video interface, an audio (speaker) interface, a wireless interface including an RF interface, and an optical interface. Such energy source output interfaces 422 may be in data communication with any number of display or communication devices including, without limitation, LEDs, LED displays, LCD displays, plasma displays, audio annunciators, and speakers. The display or communication devices may be configured to provide information to a user regarding the use of the medical device energy source 220a including an amount of electrical energy sourced by the energy source 435 during a medical procedure, an indication of a fault condition of the energy source 435, or an indication that the medical device 210 is not an appropriate device to receive electrical energy from the energy source 435.

The energy source data bus 430 may be configured to transfer data and/or information among the components of the energy source computing device 450. The energy source data bus 430 may be configured to direct instructions to the energy source 435 to cause the energy source 435 to regulate the amount of electrical energy sourced therefrom. The energy source data bus 430 may also be configured to transmit data to and receive data from the energy source data interface 224, thereby permitting exchange of data with the medical device 210a. The energy source data interface 224 may thus be in operative communication with the energy source processor unit 410 by means of the energy source data bus 430. The energy source data bus 430 may further be configured to transmit data to and receive data from the energy source network communication interface 228, thereby permitting exchange of data with a medical device network server 230. The energy source network communication interface 228 may thus be in operative communication with the energy source processor unit 410 by means of the energy source data bus 430.

It may be recognized that the energy source processor unit 410 may include similar devices as those disclosed above with respect to the medical device processor unit 310. Additionally, the one or more energy source memory storage components 412 may include similar devices as those disclosed above with respect to the medical device memory storage components 312. Further, the energy source data bus 430 may include similar devices as those disclosed above with respect to the device data bus 330. It may also be recognized that the energy source data interface 224 may include complementary components to those disclosed above with respect to the device data interface 218. In some non-limiting examples, the energy source data interface 224 may comprise one or more of a serial data interface, a parallel data interface, a wireless interface, and an optical interface

The energy source network communication interface 228 may comprise any interface configured to permit information exchange 246 with one or more networked server devices, such as medical device network server 230. The medical device network server 230 may be logically connected to the medical device energy source 220a through the energy source network communication interface 228. The energy source network communication interface 228 may encompass any known interface including, without limitation, a wired internet interface, a wireless internet interface, a WiFi interface, a BlueTooth interface, a LAN interface, a WAN interface, a telephonic interface, a cellular interface, and an optical interface. The communication interface may permit communication among networks such as local-area networks (LAN) and wide area networks (WAN). Non-limiting examples of LAN technologies may include Fiber Distributed Data Interface (FDDI), Copper Distributed Data Interface (CDDI), Ethernet/IEEE 802.3, Token Ring/IEEE 802.5 and the like. Non-limiting examples of WAN technologies may include, but are not limited to, point-to-point links, circuit switching networks like Integrated Services Digital Networks (ISDN) and variations thereon, packet switching networks, and Digital Subscriber Lines (DSL).

The energy source network communication interface 228 may further comprise wireless or cellular communications interfaces. Examples of wireless protocols may include various wireless local area network (WLAN) protocols, including the Institute of Electrical and Electronics Engineers (IEEE) 802.xx series of protocols, such as IEEE 802.11a/b/g/n, IEEE 802.16, IEEE 802.20, and so forth. Other examples of wireless protocols may include various wireless wide area network (WWAN) protocols, such as GSM cellular radiotelephone system protocols with GPRS, CDMA cellular radiotelephone communication systems with 1×RTT, EDGE systems, EV-DO systems, EV-DV systems, HSDPA systems, and so forth. Further examples of wireless protocols may include wireless personal area network (PAN) protocols, such as an Infrared protocol, a protocol from the Bluetooth Special Interest Group (SIG) series of protocols, including Bluetooth Specification versions v1.0, v1.1, v1.2, v2.0, v2.0 with Enhanced Data Rate (EDR), as well as one or more Bluetooth Profiles, and so forth. Yet another example of wireless protocols may include near-field communication techniques and protocols, such as electro-magnetic induction (EMI) techniques. An example of EMI techniques may include passive or active radio-frequency identification (RFID) protocols and devices. Other suitable protocols may include Ultra Wide Band (UWB), Digital Office (DO), Digital Home, Trusted Platform Module (TPM), ZigBee, and so forth.

Examples of cellular communication systems may include CDMA cellular radiotelephone communication systems, GSM cellular radiotelephone systems, North American Digital Cellular (NADC) cellular radiotelephone systems, Time Division Multiple Access (TDMA) cellular radiotelephone systems, Extended-TDMA (E-TDMA) cellular radiotelephone systems, Narrowband Advanced Mobile Phone Service (NAMPS) cellular radiotelephone systems, third generation (3G) systems such as WCDMA, CDMA-2000, UMTS cellular radiotelephone systems compliant with the Third-Generation Partnership Project (3GPP), and so forth.

As disclosed above, FIG. 4A depicts a medical device energy source 220a having an energy source power interface 226a suitable for conducting electrical energy to a corded medical device 210a via an attached power cable 244. FIG. 4B depicts a portion of a medical device energy source 220b having an energy source power interface 226b configured to provide electrical energy to a cordless medical device 210b which may store the power in a battery 350. The medical device energy source 220b may include an energy source power interface 226b such as a docking station that may be complementary to the device power interface 217b of the cordless medical device 210b. The energy source power interface 226b may also receive electrical energy from energy source 435 via energy source device power bus 420. It may be understood that the components and operations disclosed above with respect to medical device energy source 220a and its components as depicted in FIG. 4A may also be similar to those in the medical device energy source 220b as depicted in FIG. 4B (with the exception of the energy source power interface 226a). In one non-limiting example, medical device energy source 220b may be configured to exchange data with the medical device 210b through the energy source data interface 224 over a data cable 242 in communication with the medical device data interface 218. In another non-limiting example, the energy source data interface 224 of medical device energy source 220b may communicate data with the medical device data interface 218 over a wireless interface. It may be recognized that wireless communication between medical device energy source 220b and medical device 210b may result in medical device 210b having no physical attachments to medical device energy source 220b, permitting unencumbered used of medical device 210b.

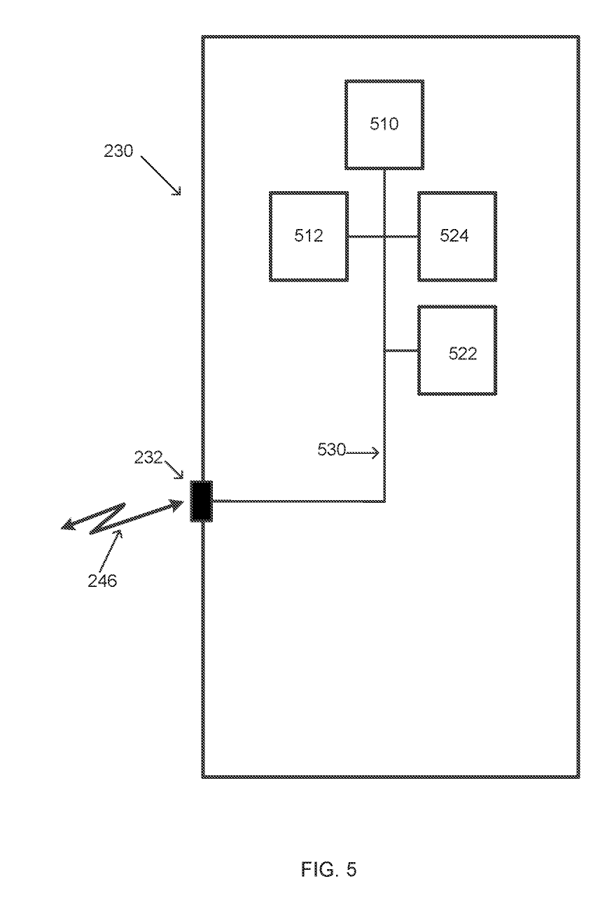

FIG. 5 depicts a block diagram of an example of a medical device network server 230. The medical device network server 230 may be composed of a medical device network server processor unit 510, one or more medical device network server memory storage components 512, one or more medical device network server input interfaces 524, one or more medical device network server output interfaces 522, and a medical device network server data bus 530. The medical device network server 230 may have a network server communication interface 232. The medical device network server processor unit 510 may be in operative communication with the one or more medical device network server memory storage components 512 and the network server communication interface 232 via the network server data bus 530. The medical device network server 230 may store one or more databases of information related to the medical device 210a,b, the energy source 220a,b, and their respective functions. The database may be stored in the one or more medical device network server memory storage components 512. The one or more medical device network server memory storage components 512 may also include instructions that may cause the medical device network sever processor unit 510 to operate according to those instructions. In some non-limiting examples, medical device network server memory storage components 512 may include instructions: to receive a request for database data from a medical device energy source; to transmit to the medical device energy source all or a portion of data from the database; to update the status indicator of a medical device in the database according to the medical device identity code; and to update usage data of a medical device in the database according to the medical device identity code. The medical device network server 230 may be in data communication with the energy source 220a,b via a network server communication interface 232 and an energy source network communication interface 228. Data communication 246 between the medical device network server 230 and the energy source 220a,b may be accomplished through any standard data exchange method. The network server communication interface 232 may comprise interfaces and protocols complementary to those disclosed above with respect to the energy source network communication interface 228.

It may be recognized that the medical device network server processor unit 510 may include similar devices as those disclosed above with respect to the medical device processor unit 310. Additionally, the one or more medical device network server memory storage components 512 may include similar devices as those disclosed above with respect to the medical device memory storage components 312. Further, the medical device network server data bus 530 may include similar devices as those disclosed above with respect to the device data bus 330. It may also be recognized that the medical device network server input interfaces 524 and one or more medical device network server output interfaces 522 may include similar devices and data exchange protocols as those disclose above with respect to the energy source input interface 424 and energy source output interface 422, respectively.

The one or more medical device network server memory storage components 512 may be used to store instructions that may be executed by the medical device network server processor unit 510. The instructions may include any suitable type of code, such as source code, compiled code, interpreted code, executable code, static code, dynamic code, and the like. The instruction or a set of instructions may include those that, if executed by the medical device network server processor unit 510, may cause the medical device network server processor unit 510 to perform a method and/or operations in accordance with the embodiments. The instructions may be implemented using any suitable high-level, low-level, object-oriented, visual, compiled and/or interpreted programming language, such as C, C++, Java, BASIC, Perl, Matlab, Pascal, Visual BASIC, assembly language, machine code, and so forth.

A medical device network server input interface 524 may be in data communication with any number of user actuators including, without limitation, push buttons, slide buttons, keyboards, knobs, touch screens, and computer mice. A user may employ the actuators to direct the operation of the medical device network server 230, for example to input or modify a database that may be stored in the medical device network server memory storage components 512.

The medical device network server output interface 522 may comprise an interface configured to provide information to a user of the medical device network server 230 via medical device network server data bus 530. Non-limiting examples of such an output interface 522 may include a serial interface, a parallel interface, a video interface, an audio (speaker) interface, a wireless interface including an RF interface, and an optical interface. Such medical device network server output interfaces 522 may be in data communication with any number of display or communication devices including, without limitation, LEDs, LED displays, LCD displays, plasma displays, audio annunciators, and speakers. The display or communication devices may be configured to provide information to a user regarding the status of the medical device network server 230 or information relevant to the database stored in the medical device network server memory storage components 512.

As disclosed above, it may be recognized that proper control of the electrical energy supplied to an electrosurgical device may be critical for safe and effective operation of the device. It is therefore desirable for a medical device energy source to supply an appropriate amount of electrical energy to an electrosurgical device to promote a safe and effective therapeutic outcome. An example of a medical device system (200, FIG. 2) that may be used towards this end is one in which a medical device energy source (220, FIG. 2) may obtain information from a medical device (210, FIG. 2) and compare that information with data maintained in a database stored in a medical device network server (230, FIG. 2). As a result of that comparison, the medical device energy source may determine, based on software instructions stored in the energy source memory storage component (412, FIG. 4A), an amount of electrical energy to supply to the medical device. The medical device energy source may receive from the medical device an identifier code via the energy source data interface (224, FIG. 2). The medical device energy source may also receive (246, FIG. 2) data from the medical device network server via an energy source network communication interface (228, FIG. 2). These data may include data from a database stored in the medical device network server memory storage component 512.

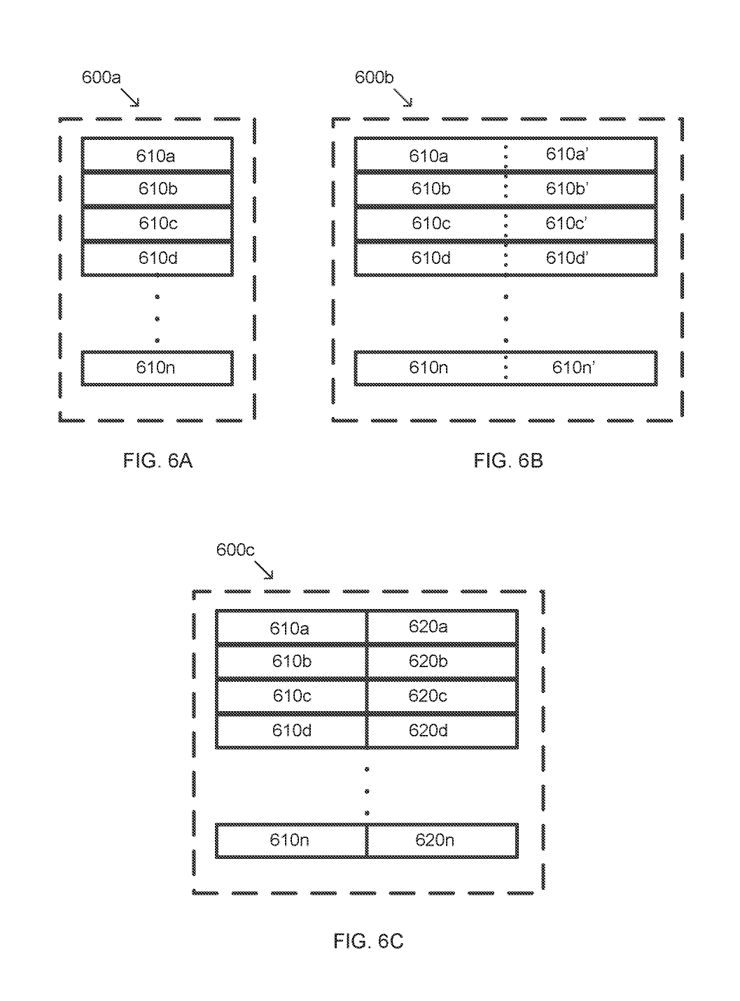

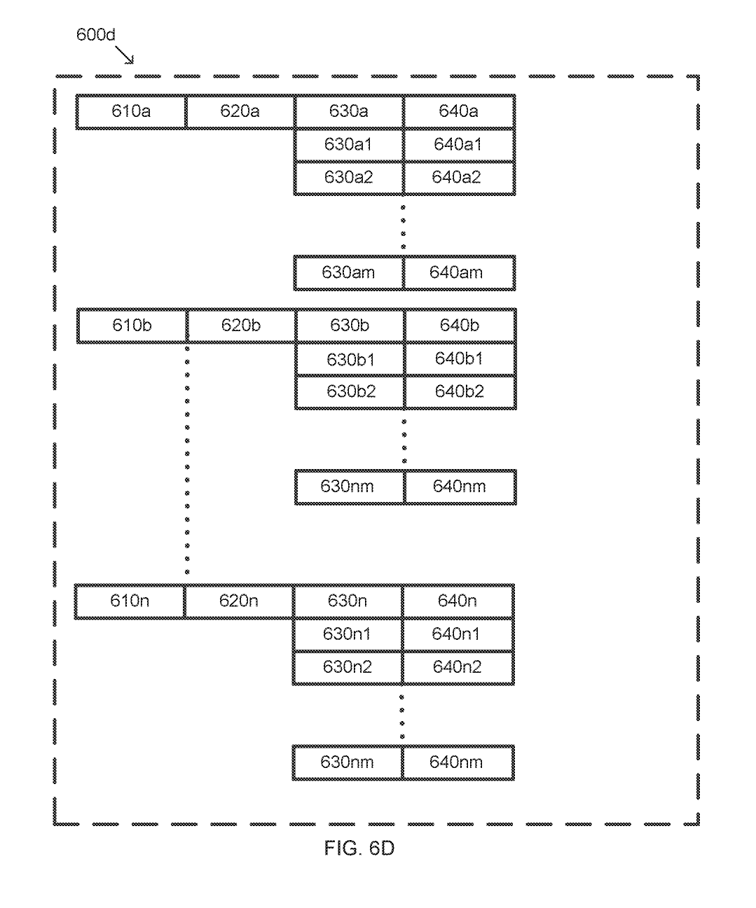

FIGS. 6A-6D depict exemplary structures of the database. It may be recognized that the examples depicted in FIGS. 6A-6D are non-limiting, and that the database may have any appropriate structure for maintaining and organizing the data.

FIG. 6A depicts a database 600a comprising a list of identity codes 610a-610n, in which each identity code is associated with a separate medical device. The number of identity codes 610n that may be stored in the database 600a may include any finite number of identity codes. For example, the number of identity codes may range from 1 identity code to about 100 identity codes. Non-limiting examples of the number of identity codes that may be including in the database 600a may include 1 identity code, 2 identity codes, 5 identity codes, 10 identity codes, 20 identity codes, 50 identity codes, 100 identity codes, or ranges in values therebetween including endpoints. As depicted in FIG. 6A, these identity codes 601a-610n may each comprise a single string of processor readable characters. Each string may include any number of processor readable characters, which may range from about 8 processor readable characters to about 256 processor readable characters. Non-limiting examples of the number of processor readable characters may include 8 processor readable characters, 16 processor readable characters, 32 processor readable characters, 64 processor readable characters, 128 processor readable characters, 256 processor readable characters, or ranges in number of processor readable characters therebetween including endpoints. Although the number of processor readable characters disclosed above are powers of 2 (that is, 2ⁿ, where n has an integer value of 3 to 8), it may be recognized that the number of processor readable characters that comprise an identity code may include any finite integer number of processor readable characters. In one non-limiting example, each string of processor readable characters that comprises an identity code 601a-610n may comprise a random or pseudo-random string of processor readable characters. In another non-limiting example, each string of processor readable characters that comprises an identity code 601a-610n may comprise a string of processor readable characters that encode data related to one of several medical devices. Examples of data that may be suitable for such encoding may include, without limitation, a medical device name, a medical device model number, a medical device serial number, a medical device date of manufacture, a medical device expiration date, or combination or combinations thereof.

FIG. 6B depicts a database 600b comprising a list of identity codes 610a-610n in which each identity code is associated with a separate medical device. The number of such identity codes 610a-610n in database 600b may be similar to the number of identity codes as disclosed above with respect to database 600a (FIG. 6A). As depicted in FIG. 6B, these identity codes 610a-610n may each comprise two individual strings (generically denoted as 610x and 610x′) of processor readable characters. Thus, each identity code may comprise a first string 610a, 610b . . . 610n and a second string 610a′, 610b′ . . . 610n′, respectively. It may be understood that although FIG. 6B depicts a database 600b composed of identity codes each having two separate strings of processor readable characters, the number of strings of processor readable characters for each identity code is not limiting, and may include 2 strings, 3 strings, 4 strings, or any finite number of strings of processor readable characters. Each string of processor readable characters that comprises an identity code in database 600b may be characterized in a similar manner as disclosed above with respect to the identity codes in database 600a (FIG. 6A). It may further be recognized the each of the two individual strings (generically denoted as 610x and 610x′) comprising the identity codes may have the same number of processor readable characters or a different number of processor readable characters. Similarly, an identifier code stored in the medical device memory component may comprise multiple strings each string having the same number of processor readable characters or a different number of processor readable characters.

FIG. 6C depicts a database 600c comprising a set of two data fields, each of which may be associated with a separate medical device. Thus, for example, a first medical device may be associated with data fields 610a and 620a, a second medical device may be associated with data fields 610b and 620b, and similar for a medical device associated with data fields 610n and 620n. The number of such paired data fields (610a,620a) through (610n,620n) in database 600c may be similar to the number of identity codes as disclosed above with respect to database 600a (FIG. 6A). In some non-limiting examples, the first data field, comprising data 610a, 610b, . . . 610n, may comprise an identity code associated with a medical device characterized as above with respect to FIG. 6A (database 600a) and 6B (database 600b).

The second data field, comprising data 620a, 620b, . . . 620n, may be associated with a second characterizer of the medical device with which the database entry is associated. In one non-limiting example, the second data field may include an indicator of a medical device status. The indicator may include a text descriptor of the status or one or more processor readable characters that may encode the status. Examples of such status indicators may include, without limitation, “NEW,” “UNUSED,” “USED,” or “REFURBISHED.” Such status indicators may be used to identify the device as being new (for example, new out of the box), an unused device (previously attached to the energy source, but not used in any medical procedure), a device used in a medical procedure, and a device that had been used but was then refurbished (for example sterilized, cleaned, mechanically adjusted) for potential reuse. It may be understood that additional or alternative status indicators may also be included. Alternatively, a second data field may include alternative characterizations of the medical device including, without limitation, a device product number, a device serial number, a device lot number, an expiration date, or any other characterization of the medical device. Although database 600c is depicted as comprising two sets of data fields associated with each medical device, it may be recognized that the database 600c may include any number of sets of data fields associated with each medical device, including 3 sets, 4 sets, or any finite number of sets of data fields.

FIG. 6D depicts a more complex database 600d. Database 600d may comprise multiple data fields (610a-610n, 620a-620n, 630a-630n, and 640a-640n), in which an entry in each data field is associated with a specific medical device. Some of the fields (for example data field 610 and 620) may comprise a single data entry for each associated medical device, for example a medical device identity code (610a) and a medical device status indicator (620a). Other data fields (630a-n and 640a-n) may include multiple data entries for each medical device stored in sub-fields. As depicted in FIG. 6D, data field 630a may include sub-fields such as 630a1, 630a2, . . . , 630am and data field 640a may include sub-fields such as 640a1, 640a2, 630am, wherein m has a finite integer value and represents the number of sub-field entries.

Data fields comprising sub-fields may be useful to retain historical data related to the use of a medical device. For example, a data field may include a total number of allowed uses of a device (630a), such as 5 total uses, and each subfield may include an individual use number, such as the number 1 in sub-field 630a1, the number 2 in sub-field 630a2, and so forth. Other types of data may include a total power permitted to be sourced to a medical device (for example, in data field 640a) as well as the amount of power sourced to the device for each use, for a first use in data field 640a1, for a second use in data field 640a2, and so forth. Other exemplary information that may be stored in such data fields comprising sub-fields may include a total time of medical device use, a maximum amount of time that the medical device may be used, and an amount of time for each use. It may be recognized that the examples of data stored in the data fields and sub-fields are not limiting, but may include any data related to the use of a given medical device.

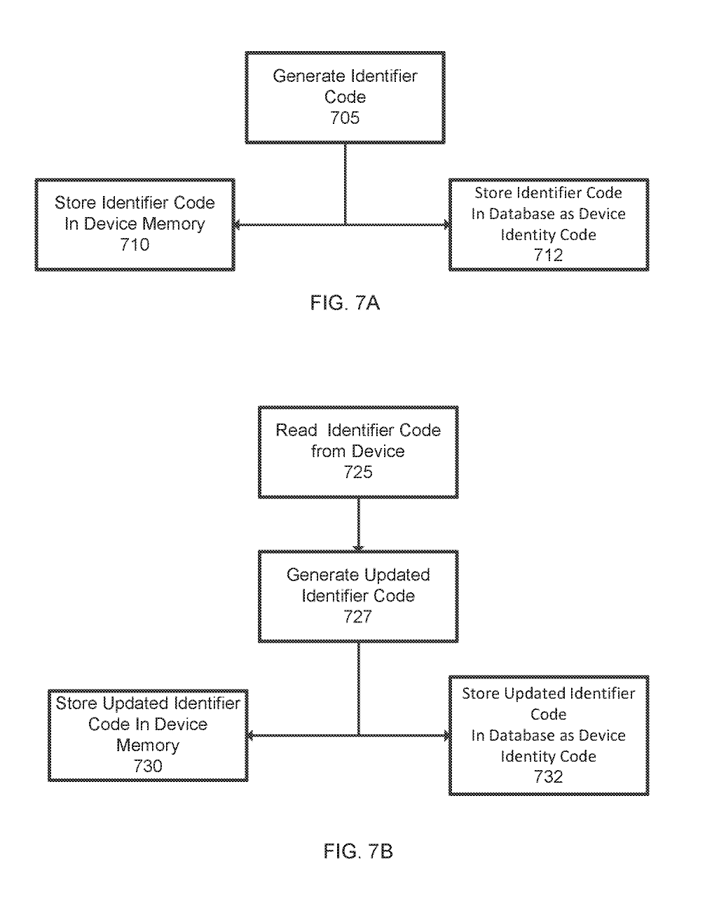

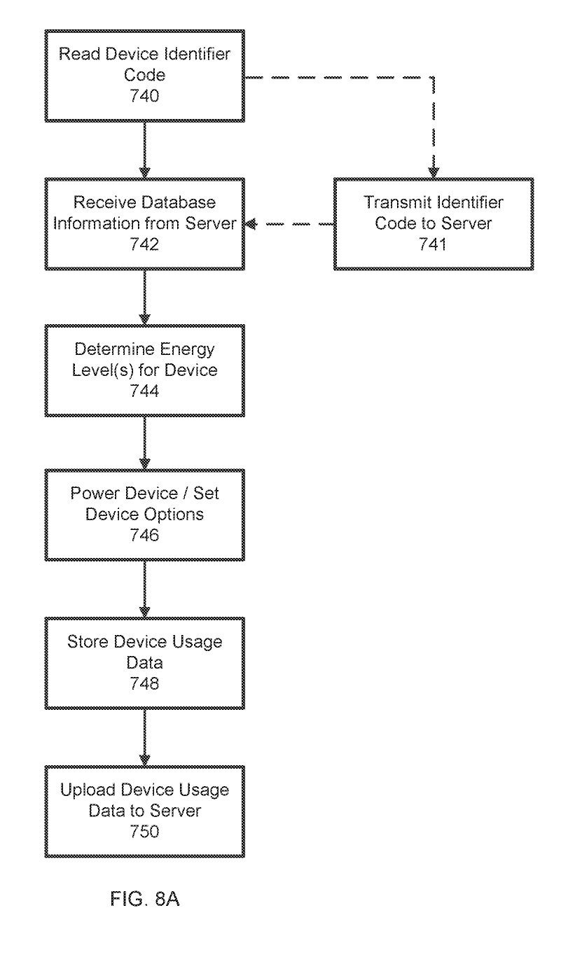

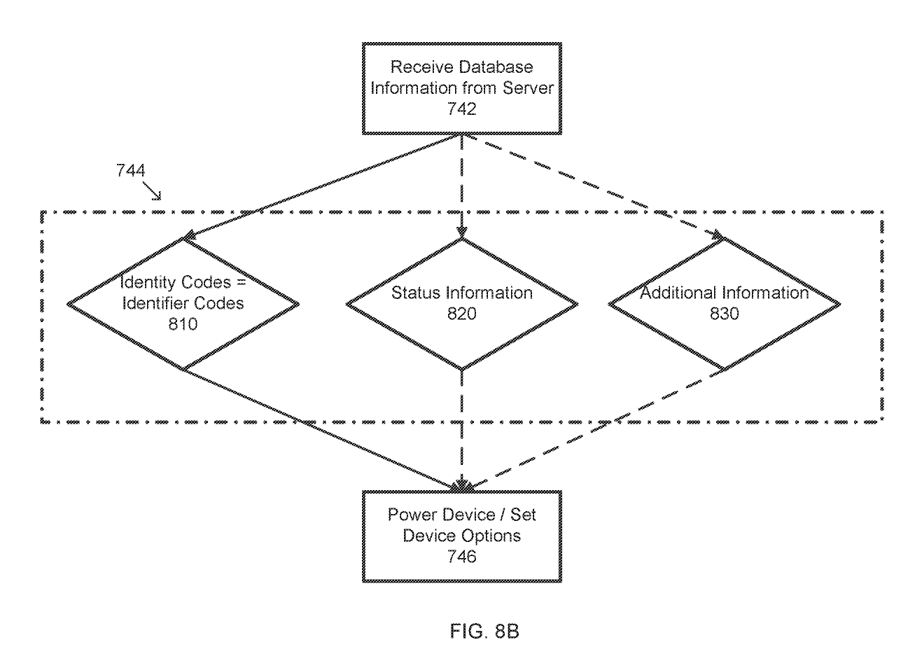

FIGS. 7A, 7B, 8A, and 8B are flow diagrams of methods related to the use of the medical device system disclosed herein. In one non-limiting example, these methods consider a medical device fabricated at a manufacturer's facility (or a facility of a third party approved by the manufacturer) which may be sold or leased to an end user such as a medical professional or a health care facility. The device may be a single use device or a multi-use (re-usable) device. After each medical use, a multi-use device may require maintenance that may include, without limitation, physical cleaning, sterilization, functional recertification (for example that moving parts operate correctly, or that electrical contacts to tissue meet required electrical specifications), or combinations thereof. The maintenance may be carried out at a facility owned and/or operated by the manufacturer or a facility owned and/or operated by a third party that is approved and certified by the manufacturer.

FIG. 7A is a flow diagram of one non-limiting example of a method related to an initial manufacture of a medical device. As part of the manufacturing process, a unique identifier code may be generated 705 for each medical device. As disclosed above, the identifier code may comprise one or more strings of processor readable characters of a defined length, and may comprise random characters or characters that encode characterizing information about the medical device. The identifier code may then be stored in the device memory storage component 710. Additionally, the identifier code may be programmed into the database stored in the medical device network server, for example as a new database entry, as a medical device identity code 712.

Depending on the database structure (see, for example, FIGS. 6A-6D), additional information may be added to the database as part of the device manufacturing process. For example, the database may include a status field, which may be programmed with an appropriate entry (such as “NEW”). Data associated with device history such as the maximum number of allowed uses, maximum energy to be supplied by the device, and maximum time for the device to be actively used may also be entered into the appropriate data fields and sub-fields. Further, additional fields in the database that may characterize the device—model number, lot number, serial number, date of manufacture, and expiration date—may be populated with data appropriate to the newly manufactured device.

It may be noted that the medical device network server may be under the sole control of the manufacturer. In one non-limiting example, the database stored in the medical device network server may be accessible to only a limited number of employees of the manufacturer. In another non-limiting example, employees of a certified or licensed third party (for example, a third party contracted to refurbish or recertify a medical device) may also have access to the database. It may be understood that software instructions stored in the network server memory storage component may be used to limit or control access to the database by the manufacturer or third party employees according to protocols known in the art.

FIG. 7B is a flow diagram of one non-limiting example of a method related to a maintenance procedure for a multi-use medical device. In one non-limiting example, the identifier code of a particular medical device may be retained after the maintenance procedure has been completed. In such an example, the equivalent identity code in the database may be retained. In an alternative non-limiting example, the identifier code of a device may be read 725 from the device memory storage component and a new or updated identifier code may be generated 727. The new or updated identifier code may be stored in the device memory storage component 730. Similarly, the new or updated identifier code may be stored in the database as a new or updated identity code 732. In one non-limiting example, the new identity code may replace the previous identity code in the database. In an alternative non-limiting example, the new identity code may be added as a new entry in the database, and the previous identity code may be retained or removed.

It may be understood, that additional data in the database may be updated, changed, or deleted as part of the maintenance procedure. For example, a status indicator associate with the medical device in the database may be set to indicate that the device has been refurbished or re-certified. Data that may be associated with the historical use of the device prior to the maintenance procedure (such as the prior number of actual uses, amount of time associated with the use of the device, and power supplied by the device) may be deleted from the database. Alternatively, the prior historical use data may be retained. Additional data related to the maintenance procedure may also be added to the database in one or more maintenance fields. Non-limiting examples of maintenance related data may include a date of maintenance, the number of times a maintenance procedure has been performed on the medical device, the name of the facility performing the maintenance, the name(s) of personnel recertifying the device, testing data associated with device re-certification, or combinations thereof.

FIG. 8A is a flow diagram of one non-limiting example of a method related to the use of a medical device system by a health care professional during a medical procedure.

A medical device may be contacted with a medical device energy source. Such contact may include affixing data cables and power cables between the two devices. Alternatively, such contact may include docking a cordless medical device with the medical device energy source, and causing a wireless data connection to be made between the two devices. The medical device energy source may be powered before the medical device is contacted with the medical device energy source, or may be powered after the medical device is contacted with the medical device energy source. Additionally, the medical device energy source, on being powered, may establish a communication link with a medical device network server over an appropriate communication channel (including one or more communication interfaces and one or more communication protocols).

After the medical device is contacted with the medical device energy source, the medical device energy source may read the device identifier code 740 from the medical device. In some non-limiting examples, the medical device may receive the device identifier code 740 via an energy source data interface. The device identifier code may be stored in a device memory storage component and may be received by the medical device energy source via an energy source data interface in operative communication with device data interface.

In one non-limiting alternative example, the medical device energy source may transmit the identifier code to the medical device network server 741. The medical device network server may compare the identifier code with one or more identity codes stored in the database. The medical device network server may respond by transmitting database information to the medical device energy source that is associated with a medical device having an identity code equal to the identifier code.

Alternatively, the medical device energy source may transmit a request to the medical device network server to receive data associated with the database. The medical device network server may respond to the request by transmitting all or a portion of the database information stored in the memory storage component of the medical device network server to the medical device energy source.

In either example, the medical device energy source may receive the database information from the server 742. The medical device energy source may include instructions that, when executed by the medical device processor, causes the medical device energy source to determine the energy level(s) to supply to the device 744 which may be based, in part, on a comparison of the device identifier code with a device identity code supplied by the medical device network server from the database.

The medical device energy source may set an appropriate power level for delivery to the medical device and/or set device options 746 of the medical device. The power level may be set by the energy source based on control instructions received from the medical device energy source computing device. As disclosed above, the energy level may comprise a therapeutic or non-therapeutic level of power. Non-limiting examples of a therapeutic amount of energy may include an amount of energy required to effect a therapy on a tissue, such as an amount of energy to cauterize a tissue, an amount of energy to shrink a tissue, or an amount of energy to cut a tissue according to the type of medical device receiving the electrical energy. Non-limiting examples of a non-therapeutic amount of energy may include an amount of energy that is not sufficient to effect a therapy on a tissue including an amount of energy to measure a tissue impedance or an amount of energy to power electronic components of the medical device. The electrical energy sourced by the energy source may be controlled with respect to a DC voltage, an AC voltage, an RMS voltage, a DC current, an AC current, an RMS current, a frequency, a pulse-width modulation, or any combination thereof.

While the medical device is being used, the medical device energy source may store some amount of medical device usage data 748 in the energy source memory storage component. Non-limiting examples of such usage data may include: a total number of times the medical device is energized with an amount of energy, the amount of energy supplied to the medical device for each energization step, the total amount of energy supplied to the medical device, the length of time the energy is supplied to the medical device for each energization step, and measurement data collected by the medical device before, during, or after each energization step. Non-limiting examples of such measurement data may include a tissue impedance value and a tissue temperature value.

The usage data obtained by the medical device energy source may be uploaded to the medical device network server 750. Such data may be uploaded during the medical procedure in which the medical device is being used or after the use of the medical device. Additional information may be uploaded to the medical device network server including, without limitation, a time stamp, a date stamp, a facility identifier (identifying the facility in which the medical device is used), and/or any other additional information related to the identity of the medical device being used, the circumstances under which the device is used, and the location in which the device is used. Additionally, the medical device energy source may upload data to the medical device network server to update the database for indicators including, but not limited to, the device status. In some examples, the medical device energy source may upload data to the medical device network server to update the device status to indicate that the device is unused or used.

It may be understood that one important feature of the a method related to the use of a medical device system as disclosed above is the step of determining the energy level(s) supplied by the medical device energy source to the medical device 744. FIG. 8B is a flow chart that suggests some of the functions of the medical device energy source that may be used to make this determination. Although three specific examples of such determinations are depicted in FIG. 8B, it may be recognized that additional or alternative determinations may be made by the medical device energy source depending on a variety of information and data obtained from the medical device, the medical device network server, and/or a medical device energy source user through one or more of the energy source input devices (via the one or more energy source input interfaces).

As depicted in FIG. 8B the medical device energy source may receive database information from the medical device network server 742 via an energy source network communication interface. The medical device energy source may determine the energy level(s) to supply to the medical device 744 based at least in part on the database information. As depicted in FIGS. 6A-6D, the database may comprise one of a variety of structures depending on the type and amount of information stored therein. The determination of the energy level supplied to the medical device 744 may be based on the type of data presented by the medical device network server as determined by the database structure.

In one non-limiting example, the medical device energy source may receive data having a database structure depicted in FIGS. 6A and 6B from the medical device network server via an energy source network communication interface. The medical device energy source may receive from the medical device network server a list of identity codes that may comprise one (for example 610a-610n in FIG. 6A) or more (for example 610a, 610a′-610n, 610n′ in FIG. 6B) strings of processor readable characters as disclosed above. The medical device energy source may then compare the list of identity codes to the identifier code received by the medical device energy source from the medical device. The medical device energy source may then determine the energy level to supply to the medical device based on determining if any one of the identity codes is equal to the identifier code 810. It may be understood that, in the case in which the identity code and the identifier code each comprise multiple strings of processor readable characters, the medical device energy source will compare each of the multiple strings comprising the identity code and identifier code. In such an example, the medical device energy source may only supply a therapeutic amount of energy to the medical device if the medical device identifier code is equal to one of the identity codes listed in the database. When the medical device energy source determines that the identifier code is the same as at least one of the identity codes, the medical device energy source may deliver a therapeutic amount of energy, a non-therapeutic amount of energy, or a combination of therapeutic and non-therapeutic amount of energy to the medical device. Alternatively, when the medical device energy source determines that the identifier code is the not same as at least one of the identity codes, the medical device energy source may deliver no energy or only a non-therapeutic amount of energy to the medical device.

In another non-limiting example, the medical device energy source may receive data having a database structure depicted in FIG. 6C from the medical device network server. Such a database may include device status information 620a-620n in FIG. 6C along with the identity codes 610a-610n in FIG. 6C. The medical device energy source may determine if any one of the identity codes equal the identifier codes 810 as disclosed above. In addition, the medical device energy source may determine an amount of energy level(s) to source to the medical device 744 based on the status information 820 corresponding to a medical device identity code that is equal to the medical device identifier code, in addition to the equality of the identifier and identity codes 810. For example, the identifier code of a medical device may be the same as an identity code included in the data base, but the status of that medical device may indicate that it has been used, and therefore should not be reused. As a result, although the medical device may be a listed device in the database, the medical device energy source may include instructions not to supply a therapeutic amount of energy to the medical device because it is used and has not been refurbished. Alternatively, the identifier code of a medical device may be the same as an identity code included in the data base, and the status of that medical device may indicate that it is new, unused or refurbished. The medical device energy source may include instructions to permit an effective or therapeutic amount of energy to be supplied to devices having such status indicators.

In yet another non-limiting example, the medical device energy source may receive data from a database structure depicted in FIG. 6D. Such a database may include additional device data maintained in a plurality of database fields such as 630 (630a1-630am through 630n1-630nm) and 640 (640a-640am through 640n-640nm) along with device status information 620a-620n and the identity codes 610a-610n. The medical device energy source may determine if any one of the identity codes equal the identifier codes 810 as disclosed above. The medical device energy source may also consider the status information 820 associated with the medical device. Further, the medical device energy source may determine the amount of energy to source to the medical device based on the additional data 830.

As one example, the medical device identifier code may be listed among the acceptable medical device identity codes, and the medical device may have a status of “NEW” or “UNUSED.” However, if an attempt is made to use the medical device after an expiration date (as determined from one of the additional fields in the database), the medical device energy source may not supply a therapeutic amount of energy to the medical device. In another example, a medical device energy source may receive additional data as part of a database from the medical device network server concerning the number of times a medical device may used or the amount of energy that may be sourced to the medical device for each use of the device or a total amount of energy that may be sourced to the medical device. During the use of the medical device, the medical device energy source may track the number of uses of the device, the amount of energy supplied during each use, and the amount of time during which the device is energized. Once a use limit has been reached—for example, the number of times the device is energized, an amount of time during which the device is energized, or the total amount of energy sourced to the device—the medical device energy source may be programmed to cease sourcing additional therapeutic energy to the device.

It will be appreciated that the terms “proximal” and “distal” are used throughout the specification with reference to a clinician manipulating one end of an instrument used to treat a patient. The term “proximal” refers to the portion of the instrument closest to the clinician and the term “distal” refers to the portion located furthest from the clinician. It will further be appreciated that for conciseness and clarity, spatial terms such as “vertical,” “horizontal,” “up,” or “down” may be used herein with respect to the illustrated embodiments. However, surgical instruments may be used in many orientations and positions, and these terms are not intended to be limiting or absolute.

Various aspects of surgical instruments and robotic surgical systems are described herein. It will be understood by those skilled in the art that the various aspects described herein may be used with the described surgical instruments and robotic surgical systems. The descriptions are provided for example only, and those skilled in the art will understand that the disclosed examples are not limited to only the devices disclosed herein, but may be used with any compatible surgical instrument or robotic surgical system.

Reference throughout the specification to “various aspects,” “some aspects,” “one example,” or “one aspect” means that a particular feature, structure, or characteristic described in connection with the aspect is included in at least one example. Thus, appearances of the phrases “in various aspects,” “in some aspects,” “in one example,” or “in one aspect” in places throughout the specification are not necessarily all referring to the same aspect. Furthermore, the particular features, structures, or characteristics illustrated or described in connection with one example may be combined, in whole or in part, with features, structures, or characteristics of one or more other aspects without limitation.

While various aspects herein have been illustrated by description of several aspects and while the illustrative embodiments have been described in considerable detail, it is not the intention of the applicant to restrict or in any way limit the scope of the appended claims to such detail. Additional advantages and modifications may readily appear to those skilled in the art. For example, it is generally accepted that endoscopic procedures are more common than laparoscopic procedures. Accordingly, the present invention has been discussed in terms of endoscopic procedures and apparatus. However, use herein of terms such as “endoscopic”, should not be construed to limit the present invention to an instrument for use only in conjunction with an endoscopic tube (e.g., trocar). On the contrary, it is believed that the present invention may find use in any procedure where access is limited to a small incision, including but not limited to laparoscopic procedures, as well as open procedures.

It is to be understood that at least some of the figures and descriptions herein have been simplified to illustrate elements that are relevant for a clear understanding of the disclosure, while eliminating, for purposes of clarity, other elements. Those of ordinary skill in the art will recognize, however, that these and other elements may be desirable. However, because such elements are well known in the art, and because they do not facilitate a better understanding of the disclosure, a discussion of such elements is not provided herein.

While several aspects have been described, it should be apparent, however, that various modifications, alterations and adaptations to those embodiments may occur to persons skilled in the art with the attainment of some or all of the advantages of the disclosure. For example, according to various aspects, a single component may be replaced by multiple components, and multiple components may be replaced by a single component, to perform a given function or functions. This application is therefore intended to cover all such modifications, alterations and adaptations without departing from the scope and spirit of the disclosure as defined by the appended claims.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Various aspects of the subject matter described herein are set out in the following numbered clauses:

Clause 1. A medical device energy source, comprising:

an energy source;

an energy source power interface configured to deliver electrical energy from the energy source; and

an energy source computing device, comprising:
an energy source processor unit;
an energy source memory storage component in operative communication with the energy source processor unit;
an energy source network communication interface in operative communication with the energy source processor unit; and
an energy source data interface in operative communication with the energy source processor unit,

wherein the energy source computing device is configured to control a function of the energy source, and

wherein, the energy source memory storage component comprises instructions that, when executed by the energy source processor unit, cause the energy source computing device to:

receive an identifier code via the energy source data interface;

receive a plurality of medical device identity codes via the energy source network communication interface;

compare the identifier code with each of the plurality of medical device identity codes; and

control the function of the energy source based on the comparison of the identifier code with each of the plurality of medical device identity codes.

Clause 2. The medical device energy source of clause 1, wherein the identifier code comprises two identifier strings, each of the two identifier strings comprising a string of processor readable characters.

Clause 3. The medical device energy source of clause 2, wherein the instructions that cause the energy source computing device to compare the identifier code with each of the plurality of medical device identity codes comprises instructions that cause the energy source computing device to compare each of the two identifier strings with each of two identity strings comprising each of the medical device identity codes.

Clause 4. The medical device energy source of any one of clauses 1-3, wherein the identifier code comprises at least one identifier string comprising a string of randomly generated processor readable characters.

Clause 5. The medical device energy source of any one of clauses 1-4, wherein, the energy source memory storage component comprises instructions that, when executed by the energy source processor unit, further cause the energy source computing device to receive, via the energy source network communication interface, a plurality of medical device status indicators, each medical device status indicator corresponding to each of the plurality of medical device identity codes.

Clause 6. The medical device energy source of clause 5, wherein the instructions that cause the energy source computing device to control the function of the energy source further comprise instructions that cause the energy source computing device to control the function of the energy source base on the medical device status indicators corresponding to a medical device identity code equal to the identifier code.

Clause 7. The medical device energy source of any one of clauses 1-6, wherein the energy source memory storage component comprises instructions that, when executed by the energy source processor unit, further cause the energy source computing device to retain, in the energy source memory storage component:

an energizer value corresponding to an amount of energy supplied by the energy source;

an energizer time value corresponding to a length of time during which the energy source supplies an amount of energy;

an energizer number corresponding to a number of times the energy source supplies an amount of energy;

or combinations thereof.

Clause 8. The medical device energy source of clause 7, wherein the instructions that cause the energy source computing device to control the function of the energy source, further comprise instructions that cause the energy source computing device to control the function of the energy source based on one or more of the energizer value, the energizer time value, and the energizer number.

Clause 9. The medical device energy source of any one of clauses 1-8, wherein the energy source power interface comprises a docking station.

Clause 10. The medical device energy source of any one of clauses 1-9, wherein the energy source data interface is configured to receive data from a medical device.

Clause 11. The medical device energy source of any one of clauses 1-10, wherein the energy source data interface is configured to transmit data to a medical device.

Clause 12. The medical device energy source of any one of clauses 1-11, further comprising a user display in operative communication with the energy source processor unit.

Clause 13. The medical device energy source of any one of clauses 1-12, wherein the energy source network communication interface comprises one or more of a wired internet interface, a wireless internet interface, a WiFi interface, a BlueTooth interface, a LAN interface, a WAN interface, a telephonic interface, a cellular interface, and an optical interface.

Clause 14. The medical device energy source of any one of clauses 1-13, wherein the energy source data interface comprises one or more of a serial data interface, a parallel data interface, a wireless interface, and an optical interface.

Clause 15. A medical device system, comprising:

a medical device, comprising:
a device memory storage component configured to store an identifier code;
a device data interface in operative connection with the memory storage component; and
a device power interface configured to receive electric power from an energy source;

a medical device energy source, comprising:
the energy source;
an energy source power interface in operative communication with the device power interface and configured to deliver electrical energy from the energy source to the medical device; and
an energy source computing device, comprising:
   an energy source processor unit;
   an energy source memory storage component in operative communication with the energy source processor unit;
   an energy source network communication interface in operative communication with the energy source processor unit and configured to transmit data to and receive data from a communication network; and
   an energy source data interface in operative connection with the device data interface,
wherein the energy source computing device is configured to control a function of the energy source; and

a medical device network server, comprising:
a network server processor unit;
a network server memory storage component in operative communication with the network server processor unit and configured to store a medical device database comprising a plurality of medical device identity codes and corresponding medical device status indicators; and
a network server communication interface in operative communication with the network server processor unit and configured to transmit data to and receive data from at least one medical device power source via the communication network;

wherein, the energy source memory storage component comprises instructions that, when executed by the energy source processor unit, cause the energy source computing device to:
receive, from the device memory storage component, the identifier code;
receive, from the network server memory storage component, the plurality of medical device identity codes from the medical device database;
compare the identifier code with each of the plurality of medical device identity codes; and
control the function of the energy source based on the comparison of the at least one identifier code with the plurality of medical device identity codes.

Clause 16. The medical device system of clause 15, wherein the identifier code comprises two identifier strings, each of the two identifier strings comprising a string of processor readable characters.

Clause 17. The medical device system of clause 16, wherein the instructions that cause the energy source computing device to compare the identifier code with each of the plurality of medical device identity codes comprises instructions that cause the energy source computing device to compare each of the two identifier strings with each of two identity strings comprising each of the medical device identity codes.

Clause 18. The medical device system of any one of clauses 15-17, wherein the instructions that cause the energy source computing device to control the function of the energy source based on the comparison of the identifier code with each of the plurality of medical device identity codes comprises instructions to cause the energy source to deliver an effective amount of electrical energy via the energy source power interface to the medical device when the identifier code is equal to at least one of the plurality of medical device identity codes.

Clause 19. The medical device system of clause 18, wherein an effective amount of electrical energy comprises a therapeutic amount of energy, a non-therapeutic amount of energy, or both a therapeutic and a non-therapeutic amount of energy to the medical device.

Clause 20. The medical device system of any one of clauses 15-19, wherein the instructions that cause the energy source computing device to control the function of the energy source based on the comparison of the identifier code with each of the plurality of medical device identity codes comprises instructions to cause the energy source computing device to cause the energy source not to deliver an effective amount of electrical energy via the energy source power interface to the medical device when the identifier code does not equal any of the plurality of medical device identity codes.

Clause 21. The medical device system of clause 20, wherein the instructions to cause the energy source computing device to cause the energy source not to deliver an effective amount of electrical energy via the energy source power interface to the medical device comprises instructions to cause the energy source computing device to cause the energy source to deliver a non-therapeutic amount of energy to the medical device.

Clause 22. The medical device system of any one of clauses 15-21, wherein the energy source memory storage component comprises instructions that, when executed by the energy source processor unit, further cause the energy source computing device to determine that the medical device is in functional communication with the medical device energy source.

Clause 23. The medical device system of clause 22, wherein the instructions that cause the energy source computing device to determine that the medical device is in functional communication with the medical device energy source comprise instructions that cause the energy source computing device to determine that the energy source power interface is in operative communication with the device power interface and that the energy source data interface is in operative connection with the device data interface.

Clause 24. The medical device system of clause 22, wherein the energy source memory storage component comprises instructions that, when executed by the energy source processor unit, further cause the energy source computing device to cause the energy source not to deliver an effective amount of electrical energy via the energy source power interface to the medical device when the medical device is not in functional communication with the medical device energy source.

Clause 25. The medical device system of any one of clauses 15-24, wherein, the energy source memory storage component comprises instructions that, when executed by the energy source processor unit, further cause the energy source computing device to receive, via the energy source network communication interface, a plurality of medical device status indicators, wherein each of the plurality of medical device status indicators corresponds to each of the plurality of medical device identity codes.

Clause 26. The medical device system of clause 25, wherein the instructions that cause the energy source computing device to control the function of the energy source further comprise instructions that cause the energy source computing device to control the function of the energy source base on a medical device status indicator corresponding to a medical device identity code equal to the identifier code.

Clause 27. The medical device system of clause 25, wherein each of the plurality of medical device status indicators is chosen from a group of: “new device”, “unused device”, “used device”, and “refurbished device”.

Clause 28. The medical device system of clause 27, wherein the energy source memory storage component further comprises instructions that, when executed by the energy source processor unit, cause the energy source computing device to cause the energy source to deliver an effective amount of electrical energy via the energy source power interface to the medical device when a value of a medical device status indicator corresponding to the medical device identity code equal to the identifier code is chosen from a group of: “new device”, “unused device”, and “refurbished device”.

Clause 29. The medical device system of clause 27, wherein the energy source memory storage component further comprises instructions that, when executed by the energy source processor unit, cause the energy source computing device to cause the energy source not to deliver an effective amount of electrical energy via the energy source power interface to the medical device when a value of a medical device status indicator corresponding to the medical device identity code equal to the identifier code is “used device”.

Clause 30. The medical device system of any one of clauses 15-29, wherein the energy source memory storage component further comprises instructions that, when executed by the energy source processor unit, cause the energy source computing device to transmit, to the medical device network server, data to update a medical device status indicator corresponding to a medical device identity code equal to the identifier code.

Clause 31. The medical device system of clause 30, wherein the medical device status indicator corresponding to a medical device identity code equal to the identifier code is chosen from a group of: “unused device” and “used device”.

Clause 32. The medical device system of clause 30, wherein the network server memory storage component comprises instructions that, when executed by the network servicer processor unit, cause the network server processor unit to:

receive, from the medical device energy source, data to update a medical device status indicator corresponding to the medical device identity code equal to the identifier code; and

update the status indicator in the data base corresponding to the medical device identity code equal to the identifier code.

Clause 33. The medical device system of any one of clauses 15-32, wherein the medical device data base further comprises one or more additional indicators corresponding to each of the medical device identity codes in the medical device data base.

Clause 34. The medical device system of any one of clauses 15-33, wherein the energy source memory storage component further comprises instructions that, when executed by the energy source processor unit, cause the energy source computing device to store in the energy source memory storage component:

an indicator of total medical device uses;

an indicator, for each use of the total medical device uses, of:
an amount of power supplied by the medical device energy source to the medical device;
and a length of time during which the medical device energy source supplies the amount of energy to the medical device;

a total amount of power supplied by the medical device energy source to the medical device over the total medical device uses;

or any combination thereof.

Clause 35. The medical device system of clause 34, wherein the one or more additional indicators comprises:

an indicator of total medical device uses;

an indicator, for each use of the total medical device uses, of:
an amount of power supplied by the medical device energy source to the medical device;
and a length of time during which the medical device energy source supplies the amount of energy to the medical device;

a total amount of power supplied by the medical device energy source to the medical device over the total medical device uses;

a medical device product number;

a medical device serial number;

a medical device lot number;

a medical device manufacturing date; and

a medical device expiration date.

Clause 36. The medical device system of clause 34, wherein the energy source memory storage component further comprises instructions that, when executed by the energy source processor unit, cause the energy source computing device to:

receive, from the medical device network server, values of the one or more additional indicators corresponding to each of the medical device identity codes in the medical device data base; and

control the function of the energy source based on the value of the one or more of the additional indicators corresponding to the medical device identity code equal to the identifier code.

Clause 37. The medical device system of clause 34, wherein the network server memory storage component comprises instructions that, when executed by the network servicer processor unit, cause the network server processor unit to: receive, from the medical device energy source, values of the one or more additional indicators corresponding to each of the medical device identity codes in the medical device data base; and update the values of the one or more additional indicators corresponding to each of the medical device identity codes in the medical device data base.

Clause 38. The medical device system of any one of clauses 15-37, wherein the device memory storage component comprises one or more of a ROM component, a PROM component, an EPROM component, an EEPROM component, and an RFID component.

Clause 39. The medical device system of any one of clauses 15-38, wherein the communication network comprises one or more of a LAN, a WAN, a WiFi network, a BlueTooth network, an internet cloud network, and a cellular network.

Clause 40. A method of controlling a medical device, comprising:

receiving, by a medical device energy source via an energy source data interface in operative communication with an energy source processor unit, an identifier code from a medical device;

storing, by the medical device energy source in a memory source memory storage component in operative communication with the energy source processor unit, the identifier code;

receiving, by the medical device energy source via an energy source network communication interface in operative communication with the energy source processor unit, a plurality of medical device identity codes from a medical device network server;

comparing, by the energy source processor unit, the identifier code with each of the plurality of medical device identity codes;

controlling, by the energy source processor unit, an amount of energy delivered by the energy source via an energy source power interface to the medical device, based on the comparison between the identifier code and the plurality of medical device identity codes; and

displaying, on a user display operatively controlled by an energy source computing device comprising the energy source processor unit, information corresponding to the amount of energy delivered by the energy source to the medical device.

Clause 41. The method of clause 40, further comprising, receiving, by the medical device energy source via the energy source network communication interface, a plurality of medical device status indicators, wherein each of the plurality of medical device status indicators corresponds to each of the plurality of medical device identity codes.

Clause 42. The method of clause 41, further comprising controlling, by the energy source processor unit, an amount of energy delivered by the energy source via the energy source power interface to the medical device, based on the medical device status indicator corresponding to a medical device identity code that is equal to the identifier code.

Clause 43. The method of any one of clauses 40-42, further comprising transmitting, by the medical device energy source to the medical device, control data to control at least one function of the medical device.

Clause 44. The method of clause 43, wherein transmitting, by the medical device energy source, control data to control at least one function of the medical device comprises transmitting, by the medical device energy source, control data to control at least one function of the medical device when the identifier code does not equal any one of the plurality of medical device identity codes.

Clause 45. The method of clause 41, further comprising transmitting, by the medical device energy source to the medical device, control data to control at least one function of the medical device based on the medical device status indicators corresponding to the medical device identity code that is equal to the identifier code.

What is claimed is:

1. A medical device energy source, comprising:
an energy source;
an energy source power interface configured to deliver electrical energy from the energy source; and
an energy source computing device, comprising:
an energy source processor unit;
an energy source memory storage component in operative communication with the energy source processor unit;
an energy source network communication interface in operative communication with the energy source processor unit; and
an energy source data interface in operative communication with the energy source processor unit,
wherein the energy source computing device is configured to control a function of the energy source, and
wherein, the energy source memory storage component comprises instructions that, when executed by the energy source processor unit, cause the energy source computing device to:
receive an identifier code via the energy source data interface;
receive a plurality of medical device identity codes via the energy source network communication interface;
compare the identifier code with each of the plurality of medical device identity codes; and
control the function of the energy source based on the comparison of the identifier code with each of the plurality of medical device identity codes.

2. The medical device energy source of claim 1, wherein the identifier code comprises two identifier strings, each of the two identifier strings comprising a string of processor readable characters.

3. The medical device energy source of claim 2, wherein the instructions that cause the energy source computing device to compare the identifier code with each of the plurality of medical device identity codes comprises instructions that cause the energy source computing device to compare each of the two identifier strings with each of two identity strings comprising each of the medical device identity codes.

4. The medical device energy source of claim 1, wherein, the energy source memory storage component comprises instructions that, when executed by the energy source processor unit, further cause the energy source computing device to receive, via the energy source network communication interface, a plurality of medical device status indicators, wherein each medical device status indicator corresponding to each of the plurality of medical device identity codes.

5. The medical device energy source of claim 4, wherein the instructions that cause the energy source computing device to control the function of the energy source further comprise instructions that cause the energy source computing device to control the function of the energy source base on the medical device status indicators corresponding to a medical device identity code equal to the identifier code.

6. The medical device energy source of claim 1, wherein the energy source memory storage component comprises instructions that, when executed by the energy source processor unit, further cause the energy source computing device to retain, in the energy source memory storage component:
an energizer value corresponding to an amount of energy supplied by the energy source;
an energizer time value corresponding to a length of time during which the energy source supplies an amount of energy;
an energizer number corresponding to a number of times the energy source supplies an amount of energy;
or combinations thereof.

7. The medical device energy source of claim 1, further comprising a user display in operative communication with the energy source processor unit.

8. A medical device system, comprising:
a medical device, comprising:
a device memory storage component configured to store an identifier code;
a device data interface in operative connection with the memory storage component; and
a device power interface configured to receive electric power from an energy source;
a medical device energy source, comprising:
the energy source;
an energy source power interface in operative communication with the device power interface and configured to deliver electrical energy from the energy source to the medical device; and
an energy source computing device, comprising:
an energy source processor unit;
an energy source memory storage component in operative communication with the energy source processor unit;
an energy source network communication interface in operative communication with the energy source processor unit and configured to transmit data to and receive data from a communication network; and
an energy source data interface in operative connection with the device data interface,
wherein the energy source computing device is configured to control a function of the energy source; and
a medical device network server, comprising:
a network server processor unit;
a network server memory storage component in operative communication with the network server processor unit and configured to store a medical device database comprising a plurality of medical device identity codes and corresponding medical device status indicators; and
a network server communication interface in operative communication with the network server processor unit and configured to transmit data to and receive data from at least one medical device power source via the communication network;
wherein, the energy source memory storage component comprises instructions that, when executed by the energy source processor unit, cause the energy source computing device to:
receive, from the device memory storage component, the identifier code;
receive, from the network server memory storage component, the plurality of medical device identity codes from the medical device database;
compare the identifier code with each of the plurality of medical device identity codes; and
control the function of the energy source based on the comparison of the at least one identifier code with the plurality of medical device identity codes.

9. The medical device system of claim 8, wherein the identifier code comprises two identifier strings, each of the two identifier strings comprising a string of processor readable characters, and
wherein the instructions that cause the energy source computing device to compare the identifier code with each of the plurality of medical device identity codes comprises instructions that cause the energy source computing device to compare each of the two identifier strings with each of two identity strings comprising each of the medical device identity codes.

10. The medical device system of claim 8, wherein, the energy source memory storage component comprises instructions that, when executed by the energy source processor unit, further cause the energy source computing device to receive, via the energy source network communication interface, a plurality of medical device status indicators, wherein each of the plurality of medical device status indicators corresponds to each of the plurality of medical device identity codes, and
wherein the instructions that cause the energy source computing device to control the function of the energy source further comprise instructions that cause the energy source computing device to control the function of the energy source base on a medical device status indicator corresponding to a medical device identity code equal to the identifier code.

11. The medical device system of claim 8, wherein the energy source memory storage component further comprises instructions that, when executed by the energy source processor unit, cause the energy source computing device to transmit, to the medical device network server, data to update a medical device status indicator corresponding to a medical device identity code equal to the identifier code.

12. The medical device system of claim 11, wherein the network server memory storage component comprises instructions that, when executed by the network servicer processor unit, cause the network server processor unit to:
receive, from the medical device energy source, data to update a medical device status indicator corresponding to the medical device identity code equal to the identifier code; and
update the status indicator in the data base corresponding to the medical device identity code equal to the identifier code.

13. The medical device system of claim 8, wherein the medical device data base further comprises one or more additional indicators corresponding to each of the medical device identity codes in the medical device data base.

14. The medical device system of claim 8, wherein the energy source memory storage component further comprises instructions that, when executed by the energy source processor unit, cause the energy source computing device to store in the energy source memory storage component:
an indicator of total medical device uses;
an indicator, for each use of the total medical device uses, of:
an amount of power supplied by the medical device energy source to the medical device; and
a length of time during which the medical device energy source supplies the amount of energy to the medical device;
a total amount of power supplied by the medical device energy source to the medical device over the total medical device uses;
or any combination thereof.

15. The medical device system of claim 14, wherein the energy source memory storage component further comprises instructions that, when executed by the energy source processor unit, cause the energy source computing device to:
receive, from the medical device network server, values of the one or more additional indicators corresponding to each of the medical device identity codes in the medical device data base; and
control the function of the energy source based on the value of the one or more of the additional indicators corresponding to the medical device identity code equal to the identifier code.

16. The medical device system of claim 14, wherein the network server memory storage component comprises instructions that, when executed by the network servicer processor unit, cause the network server processor unit to:
receive, from the medical device energy source, values of the one or more additional indicators corresponding to each of the medical device identity codes in the medical device data base; and
update the values of the one or more additional indicators corresponding to each of the medical device identity codes in the medical device data base.

17. A method of controlling a medical device, comprising:
receiving, by a medical device energy source via an energy source data interface in operative communication with an energy source processor unit, an identifier code from a medical device;
storing, by the medical device energy source in a memory source memory storage component in operative communication with the energy source processor unit, the identifier code;
receiving, by the medical device energy source via an energy source network communication interface in operative communication with the energy source processor unit, a plurality of medical device identity codes from a medical device network server;
comparing, by the energy source processor unit, the identifier code with each of the plurality of medical device identity codes;
controlling, by the energy source processor unit, an amount of energy delivered by the energy source via an energy source power interface to the medical device, based on the comparison between the identifier code and the plurality of medical device identity codes; and
displaying, on a user display operatively controlled by an energy source computing device comprising the energy source processor unit, information corresponding to the amount of energy delivered by the energy source to the medical device.

18. The method of claim 17, further comprising transmitting, by the medical device energy source to the medical device, control data to control at least one function of the medical device.

19. The method of claim 17, further comprising, receiving, by the medical device energy source via the energy source network communication interface, a plurality of medical device status indicators, wherein each of the plurality of medical device status indicators corresponds to each of the plurality of medical device identity codes.

20. The method of claim 19, further comprising controlling, by the energy source processor unit, an amount of energy delivered by the energy source via the energy source power interface to the medical device, based on the medical device status indicator corresponding to a medical device identity code that is equal to the identifier code.

21. The method of claim 19, further comprising transmitting, by the medical device energy source to the medical device, control data to control at least one function of the medical device based on the medical device status indicators corresponding to the medical device identity code that is equal to the identifier code.
